SEQUENCE LISTING (2 sequences)

SEQ ID NO: 1 - protein, 84 aa, Homo sapiens
SVSEIQLMHNLGKHLNSMERVEWLRKKLQDVHNFVALGAPLAPRDAGSQRPRKKEDNVLVESHEKSLGEADKANVDVLTKAKSQ

SEQ ID NO: 2 - protein, 34 aa, Homo sapiens
SVSEIQLMHNLGKHLNSMERVEWLRKKLQDVHNF

(19) United States
(12) Patent Application Publication
(10) Pub. No.: US 2008/0119408 A1
(43) Pub. Date: May 22, 2008

(54) PTH FORMULATIONS FOR INTRANASAL DELIVERY

(75) Inventors: Henry R. Costantino, Woodinville, WA (US); Ching-Yuan Li, Bellevue, WA (US); Kristin B. Prinn, Seattle, WA (US)

Correspondence Address: NASTECH PHARMACEUTICAL COMPANY INC, 3830 MONTE VILLA PARKWAY, BOTHELL, WA, 98021-7266, US

(73) Assignee: NASTECH PHARMACEUTICAL COMPANY INC., Bothell, WA (US)

(21) Appl. No.: 11/774,501

(22) Filed: Jul. 6, 2007

Related U.S. Application Data
(60) Provisional application No. 60/806,760, filed on Jul. 7, 2006.

(51) Int. Cl. A61K 38/00; A61K 31/045; A61P 43/00; A61K 31/70

(52) U.S. Cl. 514/12; 514/23; 514/724

(57) ABSTRACT

What is described is an aqueous pharmaceutical formulation for intranasal delivery of PTH, consisting essentially of PTH(1-34) and sorbitol; or PTH(1-34), sorbitol, and a surface active agent; or PTH(1-34), sorbitol, and halogenated alkyl alcohol; or PTH(1-34), sorbitol, a surface active agent, and a halogenated alkyl alcohol.

This application claims the benefit under 35 U.S.C. § 119(e) of U.S. Provisional Application No. 60/806,760 filed Jul. 7, 2006, which is incorporated herein by reference in its entirety.

### BACKGROUND

Osteoporosis can be defined as a systemic skeletal disease characterized by low bone mass, microarchitectural deterioration of bone tissue, and increased bone fragility and susceptibility to fracture. It most commonly affects older populations, primarily postmenopausal women.

The prevalence of osteoporosis poses a serious health problem. The National Osteoporosis Foundation has estimated that 44 million people are experiencing the effects of osteoporosis or osteopenia. By the year 2010, osteoporosis will affect more than 52 million people and, by 2020, more than 61 million people. The prevalence of osteoporosis is greater in Caucasians and Asians than in African-Americans, perhaps because African-Americans have a higher peak bone mass. Women are affected in greater numbers than men because men have a higher peak bone density. Furthermore, as women age the rate of bone turnover increases, resulting in accelerated bone loss because of the lack of estrogen after menopause.

The goal of pharmacological treatment of osteoporosis is to maintain or increase bone strength, to prevent fractures throughout the patient's life, and to minimize osteoporosis-related morbidity and mortality by safely reducing the risk of fracture. The medications that have been used most commonly to treat osteoporosis include calcium, and vitamin D, estrogen (with or without progestin), bisphonates, selective estrogen receptor modulators (SERMs), and calcitonin.

Parathyroid hormone (PTH) has recently emerged as a popular osteoporosis treatment. Unlike other therapies that reduce bone resorption, PTH increases bone mass, which results in greater bone mineral density (BMD). PTH has multiple actions on bone, some direct and some indirect. PTH increases the rate of calcium release from bone into blood. The chronic effects of PTH are to increase the number of bone cells both osteoblasts and osteoclasts, and to increase the remodeling bone. These effects are apparent within hours after PTH is administered and persist for hours after PTH is withdrawn. PTH administered to osteoporotic patients leads to a net stimulation of bone formation especially in trabecular bone in the spine and hip resulting in a highly significant reduction in fractures. The bone formation is believed to occur by the stimulation of osteoblasts by PTH as osteoblasts have PTH receptors.

Parathyroid hormone (PTH) is a secreted, 84 amino acid residue polypeptide having the amino acid sequence Ser-Val-Ser-Glu-Ile-Gln-Leu-Met-His-Asn-Leu-Gly-Lys-His-LeuAsn-Ser-Met-Glu-Arg-Val-Glu-Trp-Leu-Arg-Lys-Lys-Leu-Gln-Asp-Val-His-Asn-Phe Val Ala Leu Gly Ala Pro Leu Ala Pro Arg Asp Ala Gly Ser Gln Arg Pro Arg Lys Lys Glu Asp Asn Val Leu Val Glu Ser His Glu Lys Ser Leu Gly Glu Ala Asp Lys Ala Asn Val Asp Val Leu Thr Lys Ala Lys Ser Gln (SEQ ID NO: 1). Studies in humans with certain forms of PTH have demonstrated an anabolic effect on bone, and have prompted significant interest in its use for the treatment of osteoporosis and related bone disorders.

Using the N-terminal 34 amino acids of the bovine and human hormone Ser-Val-Ser-Glu-Ile-Gln-Leu-Met-His-Asn-Leu-Gly-Lys-His-Leu-Asn-Ser-Met-Glu-Arg-Val-Glu-TrpLeu-Arg-Lys-Lys-Leu-Gln-Asp-Val-His-Asn-Phe (SEQ ID NO: 2) for example, which by all published accounts are deemed biologically equivalent to the full length hormone, it has been demonstrated in humans that parathyroid hormone enhances bone growth particularly when administered in pulsatile fashion by the subcutaneous route. A slightly different form of PTH, human PTH (1-38) has shown similar results.

PTH (1-34), also called teriparatide, is currently on the market under the brand name FORTEO®, Eli Lilly, Indianapolis, Ind. for the treatment of postmenopausal women with osteoporosis who are at high risk of fracture. This drug is administered by a once daily subcutaneous injection of 20 μg in a solution containing acetate buffer, mannitol, and m-cresol in water, pH 4. However, many people are adverse to injections, and thus become non-compliant with the prescribed dosing of the PTH. Thus, there is a need to develop an intranasal formulation of a parathyroid hormone peptide that has suitable bioavailability such that therapeutic levels can be achieved in the blood to be effective to treat osteoporosis or osteopenia. FORTEO® (Eli Lilly, U.S.), or FORSTEO (Eli Lilly, UK), is manufactured by recombinant DNA technology using an Escherichia coli strain. PTH (1-34) has a molecular weight of 4117.87 daltons. Reviews on PTH (1-34) and its clinical uses are published, including, e.g., Brixen et al., 2004; Dobnig, 2004; Eriksen and Robins, 2004; Quattrocchi and Kourlas 2004, are hereby incorporated by reference. FORTEO® is currently licensed in the U.S. and Europe (as FORSTEO). The safety of teriparatide has been evaluated in over 2800 patients in doses ranging from 5 to 100 μg per day in short term trials. Doses of up to 40 μg per day have been given for up to two years in long term trials. Adverse events associated with FORSTEO were usually mild and generally did not require discontinuation of therapy. The most commonly reported adverse effects were dizziness, leg cramps, nausea, vomiting and headache. Mild transient hypercalcemia has been reported with FORSTEO which is usually self limiting within 6 hours.

Currently FORTEO® is administered as a daily subcutaneous injection. The following Cₘₐₓ and AUC values are described for various doses of FORTEO (20 ug is the commercially approved dose).

| SC Dose | | CLF/F | AUC₀₋ₜ | Cₘₐₓ |
|---|---|---|---|---|
| (μg) | N | (L/hr) | (pg hr/ml) | (pg/ml) |
| 20 | 22 | 152.3 ± 91.2 | 165 ± 67.6 | 151.0 ± 56.9 |
| 40 | 16 | 124.3 ± 65.8 | 393 ± 161 | 256.2 ± 117.5 |
| 80 | 22 | 104.4 ± 27.9 | 816 ± 202.2 | 552.8 ± 183.6 |

It would be preferable for patient acceptability if a non-injected route of administration were available, including nasal, bucal, gastrointestinal and dermal. Teriparatide has previously been administered intranasally to humans at doses of up to 500 μg per day for 7 days in one study (Suntory News Release). Suntory Establishes Large Scale Production of recombinant human PTH₁₋₃₄ and obtains promising results from Phase 1 Clinical Trials using a Nasal Formulation. February 1999. http://www.suntory.com/news/1999-02.html (accessed 15 Apr. 2004) and in another study subjects received up to 1,000 μg per day for 3 months (Matsumoto et al., “Daily Nasal Spray of hPTH₁₋₃₄ for 3 Months Increases Bone Mass in Osteoporotic Subjects” (ASBMR 2004 presentation 1171, Oct. 4, 2004, Seattle, Wash.), no safety concerns were noted with this route.

Most PTH formulations are reconstituted from fresh or lyophilized hormone, and incorporate various carriers, excipients and vehicles. PTH formulations are often prepared in water-based vehicles such as saline, or water which is acidified typically with acetic acid to solubilize the hormone. Many reported formulations also incorporate albumin as a stabilizer (see, e.g., Reeve et al., Br. Med. J. 1980, 280:6228; Reeve et al., Lancet 1976, 1:1035; Reeve et al., Calcif. Tissue Res. 1976, 21:469; Hodsman et al., Bone Miner 1990 9(2): 137; Tsai et al., J. Clin. Endocrinol Metab. 1989, 69(5):1024; Isaac et al., Horm. Metab. Res. 1980, 12(9):487; Law et al., J. Clin. Invest. 1983, 72(3):1106; and Hulter, J. Clin. Hypertens 1986, 2(4):360). Other reported formulations incorporate an excipient such as mannitol with either lyophilized hormone or in the reconstituted vehicle. Some formulations used for human studies include a human PTH (1-34) preparation consisting of mannitol, heat inactivated human serum albumin, and caproic acid (a protease inhibitor) as an absorption enhancer (see Reeve et al., 1976, Calcif. Tissue Res., 21, Suppl., 469-477); a human PTH (1-38) preparation reconstituted into a saline vehicle (see Hodsman et al., 1991, 14(1), 67-83); and a bovine PTH (1-34) preparation in aqueous vehicle pH adjusted with acetic acid and containing albumin. The International Reference preparation for human PTH (1-84) consists of 100 ng of hormone ampouled with 250 μg human serum albumin and 1.25 mg lactose (1981), and for bovine PTH (1-84) consists of 10 μg lyophilized hormone in 0.01 M acetic acid and 0.1% w/v mannitol (see Martindale, The Extra Pharmacoepia, The Pharmaceutical Press, London, 29th ed., 1989 at p. 1338). A formulation aimed at improving the stability for a lyophilized preparation of h-PTH (1-34) is reported in EP 619 119 using a combination of sugar and sodium chloride. U.S. Pat. No. 5,496,801 describes a freeze-dried composition for the natural hormone, PTH (1-84), containing mannitol as an excipient and a citrate source as a non-volatile buffering agent.

U.S. Pat. No. 6,770,623 describes stabilized teriparatide formulations. The '623 formulations require a buffer. The buffering agent includes any acid or salt combination which is pharmaceutically acceptable and capable of maintaining the aqueous solution at a pH range of 3 to 7, preferably 3-6, e.g., acetate, tartrate, or citrate sources. The concentration of buffer may be in the range of about 2 mM to about 500 mM.

U.S. Pat. No. 5,407,911 describes the use of dipotassium glycyrrhizate as an emulsifying agent for nasal administration of PTH. Polysorbate 80 was determined to be inferior when used in the intranasal PTH formulations because it caused a precipitate and instability in the formulation.

Commercial exploitation of parathyroid hormone requires the development of a formulation that is acceptable in terms of storage stability and ease of preparation. Because it is a protein and thus far more labile than traditional small molecular weight drugs, a parathyroid hormone formulation presents challenges not commonly encountered by the pharmaceutical industry. Furthermore, like other proteins that have been formulated successfully, PTH is particularly sensitive to oxidation, deamidation, and hydrolysis, and requires that its N-terminal and C-terminal sequences remain intact in order to preserve bioactivity.

Preservatives are commonly employed in the pharmaceutical industry to limit microbial and fungal growth in multi-use formulations. The effect of preservatives on permeation of drugs across the nasal mucosa has been reported. For example, Harris et al reported on the bioavailability of desmopressin for a single nasal administration in humans. See Harris et al. (1988) J. Pharm. Sci. 77(4):337-9. According the package insert for DDAVP® Nasal Spray (Aventis) including either chlorobutanol or benzalkonium chloride as a preservative when DDAVP is administered intranasally results in a pharmacodynamic (antidiuetic) affect about one-tenth that of an equivalent dose administered by injection. The results suggest that the presence of preservative did not have a dramatic effect on the bioavailability of this peptide drug (MW=1183.34 Da).

Another example indicates that the choice of preservative does not have an impact on intranasal bioavailability. Calcitonin formulated in the presence of benzalkonium chloride (i.e., Miacalcin®) as claimed by Azria and Cavanak (U.S. Pat. No. 5,759,565), phenylethylalcohol, and benzyl alcohol (i.e., Fortical®) all show bioequivalence. A manuscript by Morimoto et al. described the permeability of model compounds 6-carboxyfluoroscein and 4300 Da molecular weight FITC-dextran in the absence or presence of 0.1 or 0.3% benzalkonium chloride. See Morimoto et al. (1998) Eur. J. Pharm. Sci. 6(3):225-30. The permeation of 6-carboxyfluoroscein was not significantly increased by the presence of benzalkonium chloride, and there was only a slight increase in permeation for a 4300 Da molecular weight FITC-dextran in the presence of benzalkonium chloride.

For intranasal administration, various permeation enhancers may be explored to improve the drug permeation (and hence bioavailability), in particular for large molecular weight drugs. Permeation enhancers reported for use in intranasal formulations include bile salts (see Pontiroli et al. (1987) Diabete. Metab. 13:441-443; Aungst et al. (1988) Pharm. Res. 5:305-308; Maitani et al. (1989) Drug Des. Deliv. 4:109-119; Donovan et al. (1990) Pharm. Res. 7:808-815; Wuthrich et al. (1994) Pharm. Res. 11:278-282; Hosoya et al. (1999) Biol. Pharm. Bull. 22:1089-1093; Hosoya et al (1999) Biol. Pharm. Bull. 22:1089-1093), polymers (e.g., poly-L-arginine (see Ohtake et al. (2002) J. Control Release. 82:263-275), gelatin (see Wang et al. (2002) J. Pharm. Pharmacol. 54:181-188), and chitosan (see Illum et al. (1994) Pharm. Res. 11:1186-1189; Dyer et al. (2002) Pharm. Res. 19:998-1008; Prego et al. (2005) J. Control. Release. 101: 151-162), lipids and surfactants (see Machida et al. (1994) Biol. Pharm. Bull. 17(10):1375-8; Coates et al. (1995) 12(3):235-9; Laursen et al. (1996) Eur. J. Endocrinol. 135(3):309-15; Mitra et al. (2000) Int. J. Pharm. 205(1-2):127-34), cyclodextrins (see Merkus et al. (1991) Pharm. Res. 8:588-592; Adjei et al. (1992) Pharm. Res. 9:244-249; Schipper et al. (1993) Pharm. Res. 10:682-686; Matsubara (1995) J. Pharm. Sci. 84:1295-1300; Marttin et al. (1998) J. Drug Target. 6:17-36), alkyl glycosides (see Pillion et al. (1994) Endocrinology 135:2386-2391; Ahsan et al. (2001) Pharm. Res. 18(12):1742-6; Pillion et al. (2002) J. Pharm. Sci. 91:1456-1462; Nakamura et al. (2002) J. Control Release. 79:147-155; Mustafa et al (2004) J. Pharm. Sci. 93:675-683), and tight junction modulating peptides (see Johnson et al. (2005) Expert Opin. Drug Deliv. 2:281-298; Chen et al. (2006) J. Pharm. Sci. 95(6):1364-71). A variety of preservatives and combinations thereof are available in the U.S. including benzalkonium chloride (e.g., azelastine hydrochloride, ipratropium bromide, beclomethasone dipropionate monohydrate, cromolyn sodium, desmopressin acetate, calcitonin, triamcinolone acetonide, cyancobalamin, nafarelin acetate, and tetrahydrozoline hydrochloride), benzethonium chloride (e.g., butorphanol tartrate), benzyl alcohol (e.g., calcitonin), chlorobutanol (e.g., desmopressin acetate), methyl parabin (e.g., nicotine), propyl paraben (e.g., nicotine), and phenethyl alcohol (e.g., calcitonin). See http://www.accessdata.fda.gov/scripts/cder/iig/index.cfm for the FDA preservative listing.

Formulating proteins is generally more difficult that formulating small molecules, because proteins are more susceptible to degradation (see Arakawa et al. (2001) Adv. Drug Del. Rev. 46:307-26, hereby incorporated by reference in its entirety). Thus, the stability of purified proteins is difficult to predict a priori and in general must be assessed on a case-by-case basis. FORTEO® is a liquid pharmaceutical formulation of teriparatide that requires a buffer for its stability. There remains a need for a storage-stable formulation of teriparatide that does not require a buffer, and is suitable for intranasal administration.

A potential issue with intranasal delivery of PTH or its analogs is local effect on nasal tissue. For example, Tanako and co-workers have described the effects of PTH locally administered to nasal cartilage cells in culture (see Takano T., et al., J. Dent. Res. 1987 January; 66(1):84-7; Takigawa M., et. al., J. Dent. Res. 1984 January; 63(1):19-22; Takano T., et. al., Nippon Kyosei Shika Gakkai Zasshi. 1983 September; 42(3):314-21).

Thus, there is a need to develop safe and effective intranasal formulations of PTH or PTH analogs that will be suitable for systemic delivery, but not cause significant local effects on the nasal tissue (i.e., not having an effect on nasal toxicity).

### BRIEF DESCRIPTION OF THE DRAWINGS

FIG. 1: Mean Plasma Concentration versus Time for Periods 1-5: (Linear Graph).

FIG. 2: Ratio of Cₘₐₓ to Mean, Low Dose PTH Formulations versus FORTEO®.

FIG. 3. Combined ABI permeability results for Calcitonin and PTH.

FIG. 4. Addition of CB to the formulations resulted in an increase in % TER reduction compared to formulations without preservative or with NaBz.

FIG. 5. The reduction in % TER was enhanced in the PS80 formulation with increasing concentration of CB.

FIG. 6. % permeation was increased in the PS80 formulations containing CB compared to formulations without preservative or with NaBz.

FIG. 7. Effect of CB in the presence of 0.1 mg/mL PS80 on % permeation in vitro.

FIG. 8. Effect of CB in the presence of 1 mg/mL PS80 on % permeation in vitro.

FIG. 9. Effect of CB in the absence of PS80 on % permeation in vitro.

FIG. 10. % permeation comparisons for CB v. PS80 formulations.

FIG. 11. % TER results for various preservatives containing PTH₁₋₃₄ formulations.

FIG. 12. % permeation data for various preservative containing PTH₁₋₃₄ formulations.

FIG. 13. % MTT data for various preservative containing PTH₁₋₃₄ formulations.

FIG. 14. % LDH data for various preservative containing PTH₁₋₃₄ formulations.

FIG. 15. Plot of PTH Dose v. AUCₗₐₛₜ/Dose for CB and NaBz containing formulations.

### DETAILED DESCRIPTION

The present disclosure describes an intranasal pharmaceutical composition comprising teriparatide (human parathyroid hormone 1-34) as an active pharmaceutical ingredient and a halogenated alkyl alcohol such as chlorobutanol to provide preservative effectiveness and permeation enhancement.

Preferably the hormone is parathyroid hormone and the mammal is a human. In a most preferred embodiment the parathyroid hormone peptide is PTH (1-34), also known as teriparatide. Tregear, U.S. Pat. No. 4,086,196, described human PTH analogues and claimed that the first 27 to 34 amino acids are the most effective in terms of the stimulation of adenylyl cyclase in an in vitro cell assay. PTH operates through activation of two second messenger systems, Gₛ-protein activated adenylyl cyclase (AC) and G_{q}-protein activated phospholipase C_{β}. The latter system results in a stimulation of membrane-bound protein kinase C (PKC) activity. The PKC activity has been shown to require PTH residues 29 to 32 (see Jouishomme et al. (1994) J. Bone Mineral Res. 9, (1179-1189). It has been established that the increase in bone growth, i.e., the effect which is useful in the treatment of osteoporosis, is coupled to the ability of the peptide sequence to increase AC activity. The native PTH sequence, PTH (1-84) (SEQ ID NO: 1), has been shown to have all of these activities.

The above described forms of parathyroid hormone are embraced by the terms “parathyroid hormone” or “PTH” or “PTH peptide” as used generically herein. The parathyroid hormones may be obtained by known recombinant or synthetic methods, such as described in U.S. Pat. No. 4,086,196 incorporated herein by reference.

Thus, the present disclosure is a method for treating osteoporosis or osteopenia in a mammal comprising transmucosally administering a formulation comprised of a PTH peptide, such that when 50 μg of the PTH is administered transmucosally to the mammal the concentration of the PTH peptide in the plasma of the mammal increases by at least 5 pmol, preferably at least 10 pmol per liter of plasma. Pharmaceutical formulations disclosed herein may contain PTH at a range of concentrations from at least about 1 mg/ml to at least about 12 mg/ml, including at least about 1 mg/ml, at least about 2 mg/ml, at least about 3 mg/ml, at least about 6 mg/ml, and at least about 12 mg/ml. Intranasal delivery-enhancing agents are employed which enhance delivery of PTH into or across a nasal mucosal surface. For passively absorbed drugs, the relative contribution of paracellular and transcellular pathways to drug transport depends upon the pKa, partition coefficient, molecular radius and charge of the drug, the pH of the luminal environment in which the drug is delivered, and the area of the absorbing surface. A intranasal delivery-enhancing agent of the present disclosure may be a pH control agent. A pH of the pharmaceutical formulation of the present disclosure is a factor affecting absorption of PTH via paracellular and transcellular pathways to drug transport. In one embodiment, a pharmaceutical formulation of the present disclosure is pH adjusted to between about pH 3.0 to about 7.0. In a further embodiment, a pharmaceutical formulation of the present disclosure is pH adjusted to between about pH 3.0 to 6.0. In a further embodiment, a pharmaceutical formulation of the present disclosure is pH adjusted to between about pH 4.0 to about 5.0. Generally, the pH is 4.0±0.3.

As noted above, the present disclosure provides improved methods and compositions for mucosal delivery of PTH peptide to mammalian subjects for treatment or prevention of osteoporosis or osteopenia. Examples of appropriate mammalian subjects for treatment and prophylaxis according to the methods of this disclosure this disclosure include, but are not restricted to, humans and non-human primates, livestock species, such as horses, cattle, sheep, and goats, and research and domestic species, including dogs, cats, mice, rats, guinea pigs, and rabbits.

In order to provide better understanding of the present disclosure, the following definitions are provided. As used herein, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, are to be understood to include any integer within the recited range, unless otherwise indicated. As used herein, “about” or “consisting essentially of” mean ±20% of the indicated range, value, or structure, unless otherwise indicated. As used herein, the terms “include” and “comprise” are used synonymously. It should be understood that the terms “a” and “an” as used herein refer to “one or more” of the enumerated components. The use of the alternative (e.g., “or”) should be understood to mean either one, both or any combination thereof of the alternatives.

In addition, it should be understood that the individual compounds, or groups of compounds, derived from the various combinations of the structures and substituents described herein, are disclosed by the present application to the same extent as if each compound or group of compounds was set forth individually. Thus, selection of particular structures or particular substituents is within the scope of the present disclosure.

“Analog” or “analogue” as used herein refers to a chemical compound that is structurally similar to a parent compound (e.g., a peptide, protein or a mucosal delivery enhancing agent), but differs slightly in composition (e.g., one atom or functional group is different, added, or removed). The analog may or may not have different chemical or physical properties than the original compound and may or may not have improved biological or chemical activity. For example, the analog may be more hydrophilic or it may have altered activity as compared to a parent compound. The analog may mimic the chemical or biological activity of the parent compound (i.e., it may have similar or identical activity), or, in some cases, may have increased or decreased activity. The analog may be a naturally or non-naturally occurring (e.g., chemically-modified, synthetic or recombinant) variant of the original compound. An example of an analog is a mutein (i.e., a protein analogue in which at least one amino acid is deleted, added, or substituted with another amino acid). Other types of analogs include isomers (enantiomers, diastereomers, and the like) and other types of chiral variants of a compound, as well as structural isomers.

“Derivative” or “derivatized” as used herein refers to a chemically or biologically modified version of a chemical compound (including an analog) that is structurally similar to a parent compound and (actually or theoretically) derivable from that parent compound. Generally, a “derivative” differs from an “analog” in that a parent compound may be the starting material to generate a “derivative,” whereas the parent compound may not necessarily be used as the starting material to generate an “analog.”

According to the present disclosure a PTH peptide also includes the free bases, acid addition salts or metal salts, such as potassium or sodium salts of the peptides, and PTH peptides that have been modified by such processes as amidation, glycosylation, acylation, sulfation, phosphorylation, acetylation, cyclization and other well known covalent modification methods.

The nasal spray product manufacturing process generally includes the preparation of a diluent for PTH (1-34) nasal spray, which includes 85% water plus the components of the nasal spray formulation without PTH. The pH of the diluent is then measured and adjusted to pH 4.0±0.3 with sodium hydroxide or hydrochloric acid, if necessary. The PTH (1-34) nasal spray is prepared by the non-aseptic transfer of ˜85% of the final target volume of the diluent to a screw cap bottle. An appropriate amount of PTH (1-34) is added and mixed until completely dissolved. The pH is measured and adjusted to pH 4.0±0.3 with sodium hydroxide or hydrochloric acid, if necessary. A sufficient quantity of diluent is added to reach the final target volume. Screw-cap bottles are filled and caps affixed. The above description of the manufacturing process represents a method used to prepare the initial clinical batches of drug product. This method may be modified during the development process to optimize the manufacturing process.

Currently marketed PTH requires sterile manufacturing conditions for compliance with FDA regulations. Parenteral administration, including PTH for injection or infusion, requires a sterile (aseptic) manufacturing process. Current Good Manufacturing Practices (GMP) for sterile drug manufacturing include standards for design and construction features (21 CFR § 211.42 (Apr. 1, 2005)); standards for testing and approval or rejection of components, drug product containers, and closures (§ 211.84); standards for control of microbiological contamination (§ 211.113); and other special testing requirements (§ 211.167). Non-parenteral (non-aseptic) products, such as the intranasal product of this disclosure, do not require these specialized sterile manufacturing conditions. As can be readily appreciated, the requirements for a sterile manufacturing process are substantially higher and correspondingly more costly than those required for a non-sterile product manufacturing process. These costs include much greater capitalization costs for facilities, as well as a more costly manufacturing cost: extra facilities for sterile manufacturing include additional rooms and ventilation; extra costs associated with sterile manufacturing include greater manpower, extensive quality control and quality assurance, and administrative support. As a result, manufacturing costs of an intranasal PTH product, such as that of this disclosure, are far less than those of a parenterally administered PTH product. The present disclosure satisfies the need for a non-sterile manufacturing process for PTH.

“Mucosal delivery-enhancing agents” are defined as chemicals and other excipients that, when added to an aqueous PTH formulation results in a formulation that produces a significant increase in transport of PTH peptide across a mucosa as measured by the maximum blood, serum, or cerebral spinal fluid concentration (Cₘₐₓ) or by the area under the curve, AUC, in a plot of concentration versus time. A mucosa includes the nasal, oral, intestinal, buccal, bronchopulmonary, vaginal, and rectal mucosal surfaces and in fact includes all mucus-secreting membranes lining all body cavities or passages that communicate with the exterior. Mucosal delivery enhancing agents are sometimes called carriers.

As used herein, mucosal delivery-enhancing agents include agents which enhance the release or solubility (e.g., from a formulation delivery vehicle), diffusion rate, penetration capacity and timing, uptake, residence time, stability, effective half-life, peak or sustained concentration levels, clearance and other desired mucosal delivery characteristics (e.g., as measured at the site of delivery, or at a selected target site of activity such as the bloodstream or central nervous system) of PTH peptide or other biologically active compound(s). Enhancement of mucosal delivery can thus occur by any of a variety of mechanisms, for example by increasing the diffusion, transport, persistence or stability of PTH peptide, increasing membrane fluidity, modulating the availability or action of calcium and other ions that regulate intracellular or paracellular permeation, solubilizing mucosal membrane components (e.g., lipids), changing non-protein and protein sulfhydryl levels in mucosal tissues, increasing water flux across the mucosal surface, modulating epithelial junctional physiology, reducing the viscosity of mucus overlying the mucosal epithelium, reducing mucociliary clearance rates, and other mechanisms.

As used herein, a “mucosally effective amount of PTH peptide” contemplates effective mucosal delivery of PTH peptide to a target site for drug activity in the subject that may involve a variety of delivery or transfer routes. For example, a given active agent may find its way through clearances between cells of the mucosa and reach an adjacent vascular wall, while by another route the agent may, either passively or actively, be taken up into mucosal cells to act within the cells or be discharged or transported out of the cells to reach a secondary target site, such as the systemic circulation. The methods and compositions of this disclosure may promote the translocation of active agents along one or more such alternate routes, or may act directly on the mucosal tissue or proximal vascular tissue to promote absorption or penetration of the active agent(s). The promotion of absorption or penetration in this context is not limited to these mechanisms.

As used herein “peak concentration (Cₘₐₓ) of PTH peptide in a blood plasma”, “area under concentration vs. time curve (AUC) of PTH peptide in a blood plasma”, “time to maximal plasma concentration (tₘₐₓ) of PTH peptide in a blood plasma” are pharmacokinetic parameters known to one skilled in the art. Laursen et al., Eur. J. Endocrinology 135:309-315 (1996). The “concentration vs. time curve” measures the concentration of PTH peptide in a blood serum of a subject vs. time after administration of a dosage of PTH peptide to the subject either by intranasal, intramuscular, or subcutaneous route of administration. “Cₘₐₓ” is the maximum concentration of PTH peptide in the blood serum of a subject following a single dosage of PTH peptide to the subject. “tₘₐₓ” is the time to reach maximum concentration of PTH peptide in a blood serum of a subject following administration of a single dosage of PTH peptide to the subject.

A “buffer” is generally used to maintain the pH of a solution at a nearly constant value. A buffer maintains the pH of a solution, even when small amounts of strong acid or strong base are added to the solution, by preventing or neutralizing large changes in concentrations of hydrogen and hydroxide ions. A buffer generally consists of a weak acid and its appropriate salt (or a weak base and its appropriate salt). The appropriate salt for a weak acid contains the same negative ion as present in the weak acid (see Lagowski, Macmillan Encyclopedia of Chemistry, Vol. 1, Simon & Schuster, New York, 1997 at p. 273-4). The Henderson-Hasselbach Equation, pH=pKa+log₁₀[A⁻]/[HA], is used to describe a buffer, and is based on the standard equation for weak acid dissociation, HA⇄H⁺+A⁻. Examples of commonly used buffer sources include the following: acetate, tartrate, or citrate.

The “buffer capacity” means the amount of acid or base that can be added to a buffer solution before a significant pH change will occur. If the pH lies within the range of pK-1 and pK+1 of the weak acid the buffer capacity is appreciable, but outside this range it falls off to such an extent as to be of little value. Therefore, a given system only has a useful buffer action in a range of one pH unit on either side of the pK of the weak acid (or weak base) (see Dawson, Data for Biochemical Research, Third Edition, Oxford Science Publications, 1986 at p. 419). Generally, suitable concentrations are chosen so that the pH of the solution is close to the pKa of the weak acid (or weak base) (see Lide, CRC Handbook of Chemistry and Physics, 86^{th} Edition, Taylor & Francis Group, 2005-2006 at p. 2-41). Further, solutions of strong acids and bases are not normally classified as buffer solutions, and they do not display buffer capacity between pH values 2.4 to 11.6.

“Non-infused administration” means any method of delivery that does not involve an injection directly into an artery or vein, a method which forces or drives (typically a fluid) into something and especially to introduce into a body part by means of a needle, syringe or other invasive method. Non-infused administration includes subcutaneous injection, intramuscular injection, intraperitoneal injection and the non-injection methods of delivery to a mucosa.

Osteoporosis is a systemic skeletal disease characterized by low bone mass, microarchitectural deterioration of bone tissue, and increased bone fragility and susceptibility to fracture. Osteopenia is a decreased calcification or density of bone, a descriptive term applicable to all skeletal systems in which the condition is noted.

Osteoporosis or osteopenia therapies and medical diagnosis include the administration of a clinically effective dose of PTH for the prevention and/or treatment of osteoporosis or osteopenia. As noted above, the instant disclosure provides improved and useful methods and compositions for nasal mucosal delivery of a PTH peptide to prevent and treat osteoporosis or osteopenia in mammalian subjects. As used herein, prevention and treatment of osteoporosis or osteopenia means prevention of the onset or lowering the incidence or severity of clinical osteoporosis by reducing increasing bone mass, decreasing bone resorption, or reducing the incidence of fractured bones in a patient.

The PTH peptide can also be administered in conjunction with other therapeutic agents such as bisphonates, calcium, vitamin D, estrogen or estrogen-receptor binding compounds, selective estrogen receptor modulators (SERMs), bone morphogenic proteins, or calcitonin.

Improved methods and compositions for mucosal administration of PTH peptide to mammalian subjects optimize PTH peptide dosing schedules. The present disclosure provides mucosal delivery of PTH peptide, formulated with one or more mucosal delivery-enhancing agents such as a nonionic surface active agent, wherein PTH peptide dosage release is substantially normalized and/or sustained for an effective delivery period of PTH peptide release ranging from about 0.1 to about 2.0 hours; about 0.4 to about 1.5 hours; about 0.7 to about 1.5 hours; or about 0.8 to about 1.0 hours; following mucosal administration. The sustained release of PTH peptide may be facilitated by repeated administration of exogenous PTH peptide utilizing methods and compositions of the present disclosure.

Improved compositions and methods for mucosal administration of PTH peptide to mammalian subjects optimize PTH peptide dosing schedules. The present disclosure provides improved mucosal (e.g., nasal) delivery of a formulation comprising PTH peptide in combination with one or more mucosal delivery-enhancing agents and an optional sustained release-enhancing agent or agents. Mucosal delivery-enhancing agents of the present disclosure yield an effective increase in delivery, for example, an increase in the maximal plasma concentration (Cₘₐₓ) to enhance the therapeutic activity of mucosally-administered PTH peptide. A second factor affecting therapeutic activity of PTH peptide in the blood plasma and CNS is residence time (RT). Sustained release-enhancing agents, in combination with intranasal delivery-enhancing agents, increase Cₘₐₓ and increase residence time (RT) of PTH peptide. Polymeric delivery vehicles and other agents and methods of the present disclosure that yield sustained release-enhancing formulations, for example, polyethylene glycol (PEG), are disclosed herein. The present disclosure provides an improved PTH peptide delivery method and dosage form for treatment or prevention of osteoporosis or osteopenia in mammalian subjects.

Within the mucosal delivery compositions and methods of this disclosure, various delivery-enhancing agents are employed which enhance delivery of PTH peptide into or across a mucosal surface. In this regard, delivery of PTH peptide across the mucosal epithelium can occur “transcellularly” or “paracellularly.” The extent to which these pathways contribute to the overall flux and bioavailability of the PTH peptide depends upon the environment of the mucosa, the physico-chemical properties the active agent, and the properties of the mucosal epithelium. Paracellular transport involves only passive diffusion, whereas transcellular transport can occur by passive, facilitated, or active processes. Generally, hydrophilic, passively transported, polar solutes diffuse through the paracellular route, while more lipophilic solutes use the transcellular route. Absorption and bioavailability (e.g., as reflected by a permeability coefficient or physiological assay), for diverse, passively and actively absorbed solutes, can be readily evaluated, in terms of both paracellular and transcellular delivery components, for any selected PTH peptide within this disclosure. For passively absorbed drugs, the relative contribution of paracellular and transcellular pathways to drug transport depends upon the pKa, partition coefficient, molecular radius and charge of the drug, the pH of the luminal environment in which the drug is delivered, and the area of the absorbing surface. The paracellular route represents a relatively small fraction of accessible surface area of the nasal mucosal epithelium. In general terms, it has been reported that cell membranes occupy a mucosal surface area that is a thousand times greater than the area occupied by the paracellular spaces. Thus, the smaller accessible area and the size- and charge-based discrimination against macromolecular permeation suggest that the paracellular route is a generally less favorable route than transcellular delivery for drug transport. Surprisingly, the methods and compositions of this disclosure provide for significantly enhanced transport of biotherapeutics into and across mucosal epithelia via the paracellular route. Therefore, the methods and compositions of this disclosure successfully target both paracellular and transcellular routes, alternatively, or within a single method or composition.

While the mechanism of absorption promotion may vary with different mucosal delivery-enhancing agents of this disclosure, useful reagents in this context will not substantially adversely affect the mucosal tissue and is selected according to the physicochemical characteristics of the particular PTH peptide or other active or delivery-enhancing agent. In this context, delivery-enhancing agents that increase penetration or permeability of mucosal tissues will often result in some alteration of the protective permeability barrier of the mucosa. For such delivery-enhancing agents to be of value within this disclosure, it is generally desired that any significant changes in permeability of the mucosa be reversible within a time frame appropriate to the desired duration of drug delivery. Furthermore, there should be no substantial, cumulative toxicity, nor any permanent deleterious changes induced in the barrier properties of the mucosa with long-term use.

Within certain aspects of this disclosure, delivery-enhancing agents for coordinate administration or combinatorial formulation with PTH peptide of this disclosure are selected from absorption promoting small hydrophilic molecules, including but not limited to, dimethyl sulfoxide (DMSO), dimethylformamide, ethanol, propylene glycol, and the 2-pyrrolidones. Alternatively, long-chain amphipathic molecules, for example, deacylmethyl sulfoxide, azone, sodium laurylsulfate, oleic acid, and the bile salts, may be employed to enhance mucosal penetration of the PTH peptide. Additionally, surfactants (e.g., nonionic surface active agents such as polysorbates) may be employed as adjunct compounds, processing agents, or formulation additives to enhance intranasal delivery of the PTH peptide. Agents such as DMSO, polyethylene glycol, and ethanol can, if present in sufficiently high concentrations in delivery environment (e.g., by pre-administration or incorporation in a therapeutic formulation), enter the aqueous phase of the mucosa and alter its solubilizing properties, thereby enhancing the partitioning of the PTH peptide from the vehicle into the mucosa.

Additional mucosal delivery-enhancing agents that are useful within the coordinate administration and processing methods and combinatorial formulations of this disclosure include, but are not limited to, mixed micelles; enamines; nitric oxide donors (e.g., S-nitroso-N-acetyl-DL-penicillamine, NOR1, NOR4—which are preferably co-administered with an NO scavenger such as carboxy-PITO or doclofenac sodium); sodium salicylate; glycerol esters of acetoacetic acid (e.g., glyceryl-1,3-diacetoacetate or 1,2-isopropylideneglycerine-3-acetoacetate); and other release-diffusion or intra- or trans-epithelial penetration-promoting agents that are physiologically compatible for mucosal delivery. Other delivery-enhancing agents are selected from a variety of carriers, bases and excipients that enhance mucosal delivery, stability, activity, or trans-epithelial penetration of the PTH peptide. These include, inter alia, cyclodextrins and β-cyclodextrin derivatives (e.g., 2-hydroxypropyl-β-cyclodextrin and heptakis(2,6-di-O-methyl-β-cyclodextrin). These compounds, optionally conjugated with one or more of the active ingredients and further optionally formulated in an oleaginous base, enhance bioavailability in the mucosal formulations of this disclosure. Yet additional delivery-enhancing agents adapted for mucosal delivery include medium-chain fatty acids, including mono- and diglycerides (e.g., sodium caprate—extracts of coconut oil, Capmul), and triglycerides (e.g., amylodextrin, Estaram 299, Miglyol 810).

The mucosal therapeutic and prophylactic compositions of the present disclosure may be supplemented with any suitable delivery-enhancing agent that facilitates absorption, diffusion, or penetration of PTH peptide across mucosal barriers. The penetration promoter may be any promoter that is pharmaceutically acceptable. Thus, in more detailed aspects of this disclosure compositions are provided that may incorporate one or more delivery-enhancing agents that promote penetration selected from sodium salicylate and salicylic acid derivatives (acetyl salicylate, choline salicylate, salicylamide); amino acids and salts thereof (e.g. monoaminocarboxlic acids such as glycine, alanine, phenylalanine, proline, hydroxyproline; hydroxyamino acids such as serine; acidic amino acids such as aspartic acid, glutamic acid; and basic amino acids such as lysine—inclusive of their alkali metal or alkaline earth metal salts); and N-acetylamino acids (N-acetylalanine, N-acetylphenylalanine, N-acetylserine, N-acetylglycine, N-acetyllysine, N-acetylglutamic acid, N-acetylproline, N-acetylhydroxyproline) and their salts (alkali metal salts and alkaline earth metal salts). Also provided as delivery-enhancing agents that promote penetration within the methods and compositions of this disclosure are substances which are generally used as emulsifiers (e.g. sodium oleyl phosphate, sodium lauryl phosphate, sodium lauryl sulfate, sodium myristyl sulfate, polyoxyethylene alkyl ethers, polyoxyethylene alkyl esters), caproic acid, lactic acid, malic acid and citric acid and alkali metal salts thereof, pyrrolidonecarboxylic acids, alkylpyrrolidonecarboxylic acid esters, N-alkylpyrrolidones, proline acyl esters, and the like.

Within various aspects of this disclosure, improved nasal mucosal delivery formulations and methods are provided that allow delivery of PTH peptide and other therapeutic agents within this disclosure across mucosal barriers between administration and selected target sites. Certain formulations are specifically adapted for a selected target cell, tissue or organ, or even a particular disease state.

In other aspects, formulations and methods provide for efficient, selective endo- or transcytosis of PTH peptide specifically routed along a defined intracellular or intercellular pathway. Typically, the PTH peptide is efficiently loaded at effective concentration levels in a carrier or other delivery vehicle, and is delivered and maintained in a stabilized form, for example, at the nasal mucosa and/or during passage through intracellular compartments and membranes to a remote target site for drug action (e.g., the blood stream or a defined tissue, organ, or extracellular compartment). The PTH peptide may be provided in a delivery vehicle or otherwise modified (e.g., in the form of a prodrug), wherein release or activation of the PTH peptide is triggered by a physiological stimulus (e.g. pH change, lysosomal enzymes). Often, the PTH peptide is pharmacologically inactive until it reaches its target site for activity. In most cases, the PTH peptide and other formulation components are non-toxic and non-immunogenic. In this context, carriers and other formulation components are generally selected for their ability to be rapidly degraded and excreted under physiological conditions. At the same time, formulations are chemically and physically stable in dosage form for effective storage.

Various additional preparative components and methods, as well as specific formulation additives, are provided herein which yield formulations for mucosal delivery of aggregation-prone peptides and proteins, wherein the peptide or protein is stabilized in a substantially pure, unaggregated form using a solubilization agent. A range of components and additives are contemplated for use within these methods and formulations. Exemplary of these solubilization agents are cyclodextrins (CDs), for example methyl-β-cyclodextrin (Me-β-CD), which selectively bind hydrophobic side chains of polypeptides. These CDs have been found to bind to hydrophobic patches of proteins in a manner that significantly inhibits aggregation. This inhibition is selective with respect to both the CD and the protein involved. Such selective inhibition of protein aggregation provides additional advantages within the intranasal delivery methods and compositions of this disclosure. Additional agents for use in this context include CD dimers, trimers and tetramers with varying geometries controlled by the linkers that specifically block aggregation of peptides and protein. Yet solubilization agents and methods for incorporation within this disclosure involve the use of peptides and peptide mimetics to selectively block protein-protein interactions. In one aspect, the specific binding of hydrophobic side chains reported for CD multimers is extended to proteins via the use of peptides and peptide mimetics that similarly block protein aggregation. A wide range of suitable methods and anti-aggregation agents are available for incorporation within the compositions and procedures of this disclosure.

Effective delivery of biotherapeutic agents via intranasal administration must take into account the decreased drug transport rate across the protective mucus lining of the nasal mucosa, in addition to drug loss due to binding to glycoproteins of the mucus layer. Normal mucus is a viscoelastic, gel-like substance consisting of water, electrolytes, mucins, macromolecules, and sloughed epithelial cells. It serves primarily as a cytoprotective and lubricative covering for the underlying mucosal tissues. Mucus is secreted by randomly distributed secretory cells located in the nasal epithelium and in other mucosal epithelia. The structural unit of mucus is mucin. This glycoprotein is mainly responsible for the viscoelastic nature of mucus, although other macromolecules may also contribute to this property. In airway mucus, such macromolecules include locally produced secretory IgA, IgM, IgE, lysozyme, and bronchotransferrin, which also play an important role in host defense mechanisms.

The coordinate administration methods of the instant disclosure optionally incorporate effective mucolytic or mucus-clearing agents, which serve to degrade, thin, or clear mucus from intranasal mucosal surfaces to facilitate absorption of intranasally administered biotherapeutic agents. Within these methods, a mucolytic or mucus-clearing agent may be coordinately administered as an adjunct compound to enhance intranasal delivery of PTH. Alternatively, an effective amount of a mucolytic or mucus-clearing agent may be incorporated as a processing agent within a multi-processing method of this disclosure, or as an additive within a combinatorial formulation of this disclosure, to provide an improved formulation that enhances intranasal delivery of biotherapeutic compounds by reducing the barrier effects of intranasal mucus.

A variety of mucolytic or mucus-clearing agents are available for incorporation within the methods and compositions of this disclosure. Based on their mechanisms of action, mucolytic and mucus clearing agents can often be classified into the following groups: proteases (e.g., pronase, papain) that cleave the protein core of mucin glycoproteins; sulfhydryl compounds that split mucoprotein disulfide linkages; and detergents (e.g., Triton X-100, Tween 20) that break non-covalent bonds within the mucus. Additional compounds in this context include, but are not limited to, bile salts and surfactants, for example, sodium deoxycholate, sodium taurodeoxycholate, sodium glycocholate, and lysophosphatidylcholine.

The effectiveness of bile salts in causing structural breakdown of mucus is in the order: deoxycholate>taurocholate>glycocholate. Other effective agents that reduce mucus viscosity or adhesion to enhance intranasal delivery according to the methods of this disclosure include, e.g., short-chain fatty acids, and mucolytic agents that work by chelation, such as N-acylcollagen peptides, bile acids, and saponins (the latter function in part by chelating Ca²⁺ and/or Mg²⁺ which play an important role in maintaining mucus layer structure).

Additional mucolytic agents for use within the methods and compositions of this disclosure include N-acetyl-L-cysteine (ACS), a potent mucolytic agent that reduces both the viscosity and adherence of bronchopulmonary mucus and is reported to modestly increase nasal bioavailability of human growth hormone in anesthetized rats (from 7.5 to 12.2%). These and other mucolytic or mucus-clearing agents are contacted with the nasal mucosa, typically in a concentration range of about 0.2 to about 20 mM, coordinately with administration of the biologically active agent, to reduce the polar viscosity and/or elasticity of intranasal mucus.

Still other mucolytic or mucus-clearing agents may be selected from a range of glycosidase enzymes, which are able to cleave glycosidic bonds within the mucus glycoprotein; α-amylase and β-amylase are representative of this class of enzymes, although their mucolytic effect may be limited. In contrast, bacterial glycosidases which allow these microorganisms to permeate mucus layers of their hosts may have a stronger effect.

For combinatorial use with most biologically active agents within this disclosure, including peptide and protein therapeutics, non-ionogenic detergents are generally also useful as mucolytic or mucus-clearing agents. These agents typically will not modify or substantially impair the activity of therapeutic polypeptides.

Because the self-cleaning capacity of certain mucosal tissues (e.g., nasal mucosal tissues) by mucociliary clearance is necessary as a protective function (e.g., to remove dust, allergens, and bacteria), it has been generally considered that this function should not be substantially impaired by mucosal medications. Mucociliary transport in the respiratory tract is a particularly important defense mechanism against infections. To achieve this function, ciliary beating in the nasal and airway passages moves a layer of mucus along the mucosa to removing inhaled particles and microorganisms.

Ciliostatic agents, within the methods and compositions of this disclosure, increase the residence time of mucosally (e.g., intranasally) administered PTH. In particular, within the methods and compositions of this disclosure, delivery is significantly enhanced in certain aspects by the coordinate administration or combinatorial formulation of one or more ciliostatic agents that function to reversibly inhibit ciliary activity of mucosal cells, to provide for a temporary, reversible increase in the residence time of the mucosally administered active agent(s). For use within these aspects of this disclosure, the foregoing ciliostatic factors, either specific or indirect in their activity, are all candidates for successful employment as ciliostatic agents in appropriate amounts (depending on concentration, duration and mode of delivery) such that they yield a transient (i.e., reversible) reduction or cessation of mucociliary clearance at a mucosal site of administration to enhance delivery of PTH peptide, analogs and mimetics, and other biologically active agents disclosed herein, without unacceptable adverse side effects.

Certain surface active agents (surfactants) are readily incorporated within the mucosal delivery formulations and methods of this disclosure as delivery-enhancing agents. These agents, which may be coordinately administered or combinatorially formulated with PTH and other delivery-enhancing agents disclosed herein, may be selected from a broad assemblage of known surface active agents. Examples of surface-active agent are nonionic polyoxyethylene ether, bile salts, sodium glycocholate, deoxycholate, derivatives of fusidic acid, sodium taurodihydrofusidate, L-α-phosphatidylcholine didecanoyl (DDPC), polysorbate 80 (also referred to as Tween, PS80, or Tween 80), polysorbate 20, a polyethylene glycol, cetyl alcohol, polyvinylpyrolidone, a polyvinyl alcohol, lanolin alcohol, and sorbitan monooleate. The mechanisms of action of these various classes of surface active agents include solubilization of a biologically active agent. For proteins and peptides which often form aggregates, the surface active properties of these delivery-enhancing agents can allow interactions with proteins so that smaller units, such as surfactant coated monomers, may be more readily maintained in solution. These monomers are presumably more transportable units than aggregates. A nonionic surface active agent has no charge group in its head. Examples of nonionic surface active agents are nonionic polyoxyethylene ether, polysorbate 80, polysorbate 20, polyethylene glycol, cetyl alcohol, polyvinylpyrolidone, polyvinyl alcohol, poloxamer F68, poloxamer F127, and lanolin alcohol.

Another potential mechanism of surface active agents may be the protection of a peptide or protein from proteolytic degradation by proteases in the mucosal environment. Both bile salts and some fusidic acid derivatives reportedly inhibit proteolytic degradation of proteins by nasal homogenates at concentrations less than or equivalent to those required to enhance protein absorption. This protease inhibition may be especially important for peptides with short biological half-lives.

The present disclosure provides a pharmaceutical composition that contains PTH in combination with delivery-enhancing agents disclosed herein formulated in a pharmaceutical preparation for mucosal delivery.

In certain aspects of this disclosure, the combinatorial formulations and/or coordinate administration methods herein incorporate an effective amount of PTH which may adhere to charged glass thereby reducing the effective concentration in the container. Silanized containers, for example, silanized glass containers, are used to store the finished product to reduce adsorption of the PTH to a glass container.

In yet additional aspects of this disclosure, a kit for treatment of a mammalian subject comprises a stable pharmaceutical composition of PTH formulated for mucosal delivery to the mammalian subject wherein the composition is effective for treating or preventing osteoporosis or osteopenia. The kit further comprises a pharmaceutical reagent bottle to contain the PTH. The pharmaceutical reagent bottle is composed of pharmaceutical grade polymer, glass or other suitable material. The pharmaceutical reagent bottle is, for example, a silanized glass bottle. The kit further comprises an aperture for delivery of the composition to a nasal mucosal surface of the subject. The delivery aperture is composed of a pharmaceutical grade polymer, glass or other suitable material. The delivery aperture is, for example, a silanized glass.

A silanization technique combines a special cleaning technique for the surfaces to be silanized with a silanization process at low pressure. The silane is in the gas phase and at an enhanced temperature of the surfaces to be silanized. The method provides reproducible surfaces with stable, homogeneous and functional silane layers having characteristics of a monolayer. The silanized surfaces prevent binding to the glass of polypeptides or mucosal delivery enhancing agents of the present disclosure.

The procedure is useful to prepare silanized pharmaceutical reagent bottles to hold PTH peptide compositions of the present disclosure. Glass trays are cleaned by rinsing with double distilled water (ddH₂O) before using. The silane tray is then be rinsed with 95% EtOH, and the acetone tray is rinsed with acetone. Pharmaceutical reagent bottles are sonicated in acetone for 10 minutes. After the acetone sonication, reagent bottles are washed in ddH₂O tray at least twice. Reagent bottles are sonicated in 0.1M NaOH for 10 minutes. While the reagent bottles are sonicating in NaOH, the silane solution is made under a hood. (Silane solution: 800 mL of 95% ethanol; 96 L of glacial acetic acid; 25 mL of glycidoxypropyltrimethoxy silane). After the NaOH sonication, reagent bottles are washed in ddH₂O tray at least twice. The reagent bottles are sonicated in silane solution for 3 to 5 minutes. The reagent bottles are washed in 100% EtOH tray. The reagent bottles are dried with prepurified N₂ gas and stored in a 100° C. oven for at least 2 hours before using.

Within the compositions and methods of this disclosure, PTH may be administered to subjects by a variety of mucosal administration modes, including by oral, rectal, vaginal, intranasal, intrapulmonary, or transdermal delivery, or by topical delivery to the eyes, ears, skin or other mucosal surfaces.

Compositions according to the present disclosure are often administered in an aqueous solution as a nasal or pulmonary spray and may be dispensed in spray form by a variety of methods known to those skilled in the art. Preferred systems for dispensing liquids as a nasal spray are disclosed in U.S. Pat. No. 4,511,069, hereby incorporated by reference. The formulations may be presented in multi-dose containers, for example in the sealed dispensing system disclosed in U.S. Pat. No. 4,511,069. Additional aerosol delivery forms may include, e.g., compressed air-, jet-, ultrasonic-, and piezoelectric nebulizers, which deliver the biologically active agent dissolved or suspended in a pharmaceutical solvent, e.g., water, ethanol, or a mixture thereof.

Nasal and pulmonary spray solutions of the present disclosure typically comprise PTH, formulated with a surface active agent, such as a nonionic surfactant (e.g., polysorbate-80), and water. In some aspects herein, the concentration of a polysorbate, (e.g., polysorbate 80) contained in a pharmaceutical formulation for intranasal (spray) administration may be in a range from less than about 1 mg/ml to less than about 50 mg/ml, including less than about 1 mg/ml, less than about 10 mg/ml, less than about 5 mg/ml, less than about 20 mg/ml, and less than about 50 mg/ml. Another embodiment of the present disclosure comprises PTH, formulated with methyl-β-cyclodextrin, EDTA, didecanoylphosphatidyl choline (DDPC), and water. In some embodiments of the present disclosure, the nasal spray solution further comprises a propellant. The pH of the nasal spray solution is optionally between about pH 3.0 and about 6.0, preferably 4.0±0.3. Other components may be added to enhance or maintain chemical stability, including preservatives, surfactants, dispersants, or gases. Suitable preservatives include, but are not limited to, phenol, methyl paraben, paraben, m-cresol, thiomersal, chlorobutanol (or other halogenated alkyl alcohol), benzylalkonium chloride, and the like. In certain aspects of this disclosure, a pharmaceutical formulation containing a preservative such as chlorobutanol, the concentration of chlorobutanol present in such formulation may be, for example, in a range from less than about 1 mg/ml to less than about 20 mg/ml, including less than about 1 mg/ml, less than about 5 mg/ml, less than about 10 mg/ml, less than about 15 mg/ml, and less than about 20 mg/ml. Suitable surfactants include, but are not limited to, oleic acid, sorbitan trioleate, polysorbates, lecithin, phosphatidyl cholines, and various long chain diglycerides and phospholipids. Suitable dispersants include, but are not limited to, ethylenediaminetetraacetic acid, and the like. Suitable gases include, but are not limited to, nitrogen, helium, chlorofluorocarbons (CFCs), hydrofluorocarbons (HFCs), carbon dioxide, air, and the like.

To formulate compositions for mucosal delivery within the present disclosure, the biologically active agent can be combined with various pharmaceutically acceptable additives, as well as a base or carrier for dispersion of the active agent(s). In addition, local anesthetics (e.g., benzyl alcohol), isotonizing agents (e.g., sodium chloride, mannitol, sorbitol), adsorption inhibitors (e.g., surfactants), solubility enhancing agents (e.g., cyclodextrins and derivatives thereof), stabilizers (e.g., serum albumin), and reducing agents (e.g., glutathione) can be included. When the composition for mucosal delivery is a liquid, the tonicity of the formulation, as measured with reference to the tonicity of 0.9% (w/v) physiological saline solution taken as unity, may be adjusted to a value at which no substantial, irreversible tissue damage is induced in the nasal mucosa at the site of administration. Generally, the tonicity of the solution is adjusted to a value of about ⅓ to about 3, more typically from about ½ to about 2, and most often about ¾ to about 1.7.

To further enhance mucosal delivery of pharmaceutical agents within this disclosure, PTH formulations may also contain a hydrophilic low molecular weight compound as a base or excipient. Such hydrophilic low molecular weight compounds provide a passage medium through which a water-soluble active agent, such as PTH, may diffuse through the base to the body surface where PTH is absorbed. The hydrophilic low molecular weight compound optionally absorbs moisture from the mucosa or the administration atmosphere and dissolves the water-soluble active peptide. The molecular weight of the hydrophilic low molecular weight compound is generally not more than 10000 and preferably not more than 3000. Exemplary hydrophilic low molecular weight compound include polyol compounds, such as oligo-, di- and monosaccarides such as sucrose, mannitol, sorbitol, lactose, L-arabinose, D-erythrose, D-ribose, D-xylose, D-mannose, trehalose, D-galactose, lactulose, cellobiose, gentibiose, glycerin, and polyethylene glycol. Other examples of hydrophilic low molecular weight compounds useful as carriers within this disclosure include N-methylpyrrolidone, and alcohols (e.g. oligovinyl alcohol, ethanol, ethylene glycol, and propylene glycol). These hydrophilic low molecular weight compounds can be used alone or in combination with one another or with other components of the intranasal formulation.

The compositions of this disclosure may alternatively contain, as pharmaceutically acceptable carriers, substances as required to approximate physiological conditions, such as tonicity adjusting agents, wetting agents and the like, for example, sodium lactate, sodium chloride, potassium chloride, calcium chloride, sorbitan monolaurate, and triethanolamine oleate. Conventional nontoxic pharmaceutically acceptable carriers can be used which include, for example, pharmaceutical grades of mannitol, lactose, starch, magnesium stearate, sodium saccharin, talcum, cellulose, glucose, sucrose, magnesium carbonate, and the like.

Therapeutic compositions for administering PTH can also be formulated as a solution, microemulsion, or other ordered structure suitable for high concentration of active ingredients. The carrier can be a solvent or dispersion medium containing, for example, water, ethanol, polyol (e.g., glycerol, propylene glycol, and liquid polyethylene glycol, and the like), and suitable mixtures thereof. Proper fluidity for solutions can be maintained, for example, by the use of a coating such as lecithin, by the maintenance of a desired particle size in the case of dispersible formulations, and by the use of surfactants. In many cases, it is desirable to include isotonic agents, for example, sugars, polyalcohols such as mannitol, sorbitol, or sodium chloride in the composition. Prolonged absorption of the biologically active agent can be brought about by including in the composition an agent which delays absorption, for example, monostearate salts, and gelatin.

Mucosal administration according to this disclosure allows effective self-administration of treatment by patients, provided that sufficient safeguards are in place to control and monitor dosing and side effects. Mucosal administration also overcomes certain drawbacks of other administration forms, such as injections, that are painful and expose the patient to possible infections and may present drug bioavailability problems. For nasal and pulmonary delivery, systems for controlled aerosol dispensing of therapeutic liquids as a spray are well known. In one embodiment, metered doses of active agent are delivered by means of a specially constructed mechanical pump valve, U.S. Pat. No. 4,511,069.

For prophylactic and treatment purposes, PTH may be administered to the subject intranasally once daily. In this context, a therapeutically effective dosage of the PTH may include repeated doses within a prolonged prophylaxis or treatment regimen that will yield clinically significant results to alleviate or prevent osteoporosis or osteopenia. Determination of effective dosages in this context is typically based on animal model studies followed up by human clinical trials and is guided by determining effective dosages and administration protocols that significantly reduce the occurrence or severity of targeted disease symptoms or conditions in the subject. Suitable models in this regard include, for example, murine, rat, porcine, feline, dog, non-human primate, and other accepted animal model subjects known in the art. Alternatively, effective dosages can be determined using in vitro models (e.g., immunologic and histopathologic assays). Using such models, only ordinary calculations and adjustments are typically required to determine an appropriate concentration and dose to administer a therapeutically effective amount of the biologically active agent(s) (e.g., amounts that are intranasally effective, transdermally effective, intravenously effective, or intramuscularly effective to elicit a desired response).

The actual dosage of biologically active agents will of course vary according to factors such as the disease indication and particular status of the subject (e.g., the subject's age, size, fitness, extent of symptoms, and susceptibility factors), time and route of administration, other drugs or treatments being administered concurrently, as well as the specific pharmacology of the biologically active agent(s) for eliciting the desired activity or biological response in the subject. Dosage regimens may be adjusted to provide an optimum prophylactic or therapeutic response. A therapeutically effective amount is also one in which any toxic or detrimental side effects of the biologically active agent are outweighed in clinical terms by therapeutically beneficial effects. A non-limiting range for a therapeutically effective amount of a PTH peptide within the methods and formulations of this disclosure is from about 0.7 μg/kg to about 25 μg/kg. To treat osteoporosis or osteopenia, an intranasal dose of PTH peptide is administered at dose high enough to promote the increase in bone mass but low enough so as not to induce any unwanted side-effects such as nausea. A preferred intranasal dose of PTH (1-34) is about 1 to about 10 μg/kg weight of the patient, most preferably about 6 μg/kg weight of the patient. In a standard dose a patient will receive about 1 to about 1000 μg, more preferably about 20 to about 800 μg, most preferably about 100 μg to about 600 μg with 300 μg being a dose that is considered to be highly effective.

Alternatively, a non-limiting range for a therapeutically effective amount of a biologically active agent within the methods and formulations of this disclosure is between about 0.001 pmol to about 100 pmol per kg body weight, between about 0.01 pmol to about 10 pmol per kg body weight, between about 0.1 pmol to about 5 pmol per kg body weight, or between about 0.5 pmol to about 1.0 pmol per kg body weight. Per administration, it is desirable to administer at least one microgram of PTH, more typically between about 10 μg and about 5.0 mg, and in certain embodiments between about 100 μg and 1.0 or about 2.0 mg to an average human subject. For certain oral applications, doses as high as about 0.5 mg per kg body weight may be necessary to achieve adequate plasma levels. It is to be further noted that for each particular subject, specific dosage regimens should be evaluated and adjusted over time according to the individual need and professional judgment of the person administering or supervising the administration of the permeabilizing peptide(s) and other biologically active agent(s).

An intranasal dose of a parathyroid hormone will range from about 1 μg to about 1000 μg of parathyroid hormone, preferably about 20 μg to about 800 μg, more preferably about 100 μg to about 600 μg with 300 μg being a dose that is considered to be highly effective. Repeated intranasal dosing with the formulations of this disclosure, on a schedule ranging from about 0.1 to 24 hours between doses, preferably between 0.5 and 24 hours between doses, will maintain normalized, sustained therapeutic levels of PTH peptide to maximize clinical benefits while minimizing the risks of excessive exposure and side effects. The goal is to mucosally deliver an amount of the PTH peptide sufficient to raise the concentration of the PTH peptide in the plasma of an individual to promote increase in bone mass.

Dosage of PTH agonists such as parathyroid hormone may be varied by the attending clinician or patient, if self administering an over the counter dosage form, to maintain a desired concentration at the target site.

In an alternative embodiment, this disclosure provides compositions and methods for intranasal delivery of PTH peptide, wherein the PTH peptide is repeatedly administered through an intranasal effective dosage regimen that involves multiple administrations of the PTH peptide to the subject during a daily or weekly schedule to maintain a therapeutically effective elevated and lowered pulsatile level of PTH peptide during an extended dosing period. The compositions and method provide PTH peptide that is self-administered by the subject in a nasal formulation between one and six times daily to maintain a therapeutically effective elevated and lowered pulsatile level of PTH peptide during about an 8 hour to 24 hour extended dosing period.

The instant disclosure also includes kits, packages and multicontainer units containing the above described pharmaceutical compositions, active ingredients, and/or means for administering the same for use in the prevention and treatment of diseases and other conditions in mammalian subjects. Briefly, these kits include a container or formulation that contains PTH in combination with mucosal delivery enhancing agents disclosed herein formulated in a pharmaceutical preparation for mucosal delivery.

The intranasal formulations of the present disclosure can be administered using any spray bottle (i.e., a bottle with an actuator, spray pump). An example of a nasal spray bottle is the, “Nasal Spray Pump w/Safety Clip”, which delivers a dose of about 0.1 mL per squirt and has a diptube length of 36.05 mm (Pfeiffer of America, Princeton, N.J.). Intranasal doses of a PTH peptide such as parathyroid hormone can range from about 0.1 μg/kg to about 1500 μg/kg. When administered in an intranasal spray, it is preferable that the particle size of the spray is between 10-100 μm (microns) in size, preferably 20-100 μm in size.

We have discovered that the parathyroid hormone peptides can be administered intranasally using a nasal spray or aerosol. This is surprising because many proteins and peptides have been shown to be sheared or denatured due to the mechanical forces generated by the actuator in producing the spray or aerosol. In this area the following definitions are useful:

1. Aerosol—A product that is packaged under pressure and contains therapeutically active ingredients that are released upon activation of an appropriate valve system.

2. Metered aerosol—A pressurized dosage form comprised of metered dose valves, which allow for the delivery of a uniform quantity of spray upon each activation.

3. Powder aerosol—A product that is packaged under pressure and contains therapeutically active ingredients in the form of a powder, which are released upon activation of an appropriate valve system.

4. Spray aerosol—An aerosol product that utilizes a compressed gas as the propellant to provide the force necessary to expel the product as a wet spray; it generally applicable to solutions of medicinal agents in aqueous solvents.

5. Spray—A liquid minutely divided as by a jet of air or steam. Nasal spray drug products contain therapeutically active ingredients dissolved or suspended in solutions or mixtures of excipients in nonpressurized dispensers.

6. Metered spray—A non-pressurized dosage form consisting of valves that allow the dispensing of a specified quantity of spray with each activation.

7. Suspension spray—A liquid preparation containing solid particles dispersed in a liquid vehicle and in the form of course droplets or as finely divided solids.

The fluid dynamic characterization of the aerosol spray emitted by metered nasal spray pumps as a drug delivery device (“DDD”). Spray characterization is an integral part of the regulatory submissions necessary for Food and Drug Administration (“FDA”) approval of research and development, quality assurance and stability testing procedures for new and existing nasal spray pumps.

Thorough characterization of the spray's geometry has been found to be the best indicator of the overall performance of nasal spray pumps. In particular, measurements of the spray's divergence angle (plume geometry) as it exits the device; the spray's cross-sectional ellipticity, uniformity and particle/droplet distribution (spray pattern); and the time evolution of the developing spray have been found to be the most representative performance quantities in the characterization of a nasal spray pump. During quality assurance and stability testing, plume geometry and spray pattern measurements are key identifiers for verifying consistency and conformity with the approved data criteria for the nasal spray pumps.

### Definitions

Plume Height—the measurement from the actuator tip to the point at which the plume angle becomes non-linear because of the breakdown of linear flow. Based on a visual examination of digital images, and to establish a measurement point for width that is consistent with the farthest measurement point of spray pattern, a height of 30 mm is defined for this study

Major Axis—the largest chord that can be drawn within the fitted spray pattern that crosses the COMw in base units (mm)

Minor Axis—the smallest chord that can be drawn within the fitted spray pattern that crosses the COMw in base units (mm)

Ellipticity Ratio—the ratio of the major axis to the minor axis

D₁₀—the diameter of droplet for which 10% of the total liquid volume of sample consists of droplets of a smaller diameter (μm)

D₅₀—the diameter of droplet for which 50% of the total liquid volume of sample consists of droplets of a smaller diameter (μm), also known as the mass median diameter

D₉₀—the diameter of droplet for which 90% of the total liquid volume of sample consists of droplets of a smaller diameter (μm)

Span—measurement of the width of the distribution, the smaller the value, the narrower the distribution. Span is calculated as:

$$\frac{( {D}_{90} - {D}_{10} )}{{D}_{50}}$$

% RSD—percent relative standard deviation, the standard deviation divided by the mean of the series and multiplied by 100, also known as % CV.

A nasal spray device can be selected according to what is customary in the industry or acceptable by the regulatory health authorities. One example of a suitable device is described in described in U.S. application Ser. No. 10/869,649 (S. Quay and G. Brandt: Compositions and methods for enhanced mucosal delivery of Y2 receptor-binding peptides and methods for treating and preventing obesity).

To treat osteoporosis or osteopenia, an intranasal dose of a PTH peptide parathyroid hormone is administered at dose high enough to promote an increase in bone mass, but low enough so as not to induce any unwanted side-effects such as nausea. A preferred intranasal dose of a PTH is about 1 μg-10 μg/kg weight of the patient, most preferably about 6 μg/kg weight of the patient. In a standard dose a patient will receive 1 μg to 1000 μg, more preferably about between 20 μg to 800 μg, most preferably 100 μg to about 600 μg with 300 μg being the dose that is considered to be highly effective. A PTH peptide such as parathyroid hormone (1-34) is preferably administered once a day.

All U.S. patents, U.S. patent application publications, U.S. patent applications, foreign patents, foreign patent applications, non-patent publications, figures, tables, and websites referred to in this specification are expressly incorporated herein by reference, in their entirety.

The following examples are provided by way of illustration, not limitation.

### EXAMPLE 1

### Reagents and Cells

The effect of various permeation enhancers on PTH formulations was measured in a MatTek cell model (MatTek, Corp. Ashland, Mass.). Three permeation enhancers (EDTA, ethanol, and polysorbate 80 (Tween 80)) were evaluated individually and in combination with one another. Sorbitol was used as a tonicifiers to adjust the osmolality of formulations to 220 mOsm/kg whenever applicable. The formulation pH was adjusted to ˜4.0. The permeation enhancer combination of 45 mg/ml Me-β-CD, 1 mg/ml DDPC, and 1 mg/ml EDTA at pH 4.5 served as the positive control. The formulation containing sorbitol only was used as the negative control. Each formulation was evaluated in the presence and absence of preservative. For all initial formulations tested, sodium benzoate was used as the preservative.

The MatTek cell line is normal, human-derived tracheal/bronchial epithelial cells (EpiAirway™ Tissue Model). Cells were cultured for 24-48 hours before using to produce a tissue insert.

Each tissue insert was placed in an individual well containing 1 ml media. On the apical surface of the inserts, 100 μl of test formulation was applied, and the samples were shaken for 1 h at 37° C. The underlying culture media samples were taken at 20, 40, and 60 minutes and stored at 4° C. for up to 48 hours for lactate dehydrogenase (LDH, cytotoxicity) and sample penetration (PTH HPLC evaluations). The 60-min samples were used for the lactate dehydrogenase (LDH, cytotoxicity) assay. Transepithelial electrical resistance (TER) was measured before and after the 1-h incubation. Following the incubation, the cell inserts were analyzed for cell viability via the mitochondrial dehydrogenase (MDH) assay.

A reverse phase high pressure liquid chromatography method was used to determine the Teriparatide concentration in the tissue permeation assay.

### EXAMPLE 2

### Transepithelial Electrical Resistance

TER measurements were accomplished using the Endohm-12 Tissue Resistance Measurement Chamber connected to the EVOM Epithelial Voltammeter (World Precision Instruments, Sarasota, Fla.) with the electrode leads. The electrodes and a tissue culture blank insert were equilibrated for at least 20 minutes in MatTek medium with the power off prior to checking calibration. The background resistance was measured with 1.5 ml Media in the Endohm tissue chamber and 300 μl Media in the blank insert. The top electrode was adjusted so that it was close to, but not in contact with, the top surface of the insert membrane. Background resistance of the blank insert was about 5-20 ohms. For each TER determination, 300 μl of MatTek medium was added to the insert followed by placement in the Endohm chamber. Resistance was expressed as (resistance measured−blank)×0.6 cm².

The formulations tested for TER reduction are described in Table 1.

TABLE 1
Description of Formulations Containing GRAS permeation Enhancers

| | Conc. (mg/ml) | | | | | | | | |
|---|---|---|---|---|---|---|---|---|---|
| | | | | | | Tween | | Sorbital | |
| Sample # | PTH | Me-β-CD | DDPC | EDTA | Ethanol | 80 | NaBz | (mg/ml) | pH |
| 1 | 7.5 | 45 | 1 | 1 | 0 | 0 | 0 | 28.8 | 4.5 |
| 2 | 7.5 | 45 | 1 | 1 | 0 | 0 | 4.75 | 16.8 | 4.5 |
| 3 | 7.5 | 0 | 0 | 1 | 0 | 0 | 0 | 34.2 | 4.0 |
| 4 | 7.5 | 0 | 0 | 1 | 0 | 0 | 3 | 26.7 | 4.0 |
| 5 | 7.5 | 0 | 0 | 0 | 0 | 0 | 0 | 35.9 | 4.0 |
| 6 | 7.5 | 0 | 0 | 0 | 0 | 0 | 3 | 28.3 | 4.0 |
| 7 | 7.5 | 0 | 0 | 0 | 10 | 0 | 0 | 0 | 4.0 |
| 8 | 7.5 | 0 | 0 | 1 | 10 | 0 | 0 | 0 | 4.0 |
| 9 | 7.5 | 0 | 0 | 10 | 10 | 0 | 0 | 0 | 4.0 |
| 10 | 7.5 | 0 | 0 | 0 | 10 | 0 | 3 | 0 | 4.0 |
| 11 | 7.5 | 0 | 0 | 1 | 10 | 0 | 3 | 0 | 4.0 |
| 12 | 7.5 | 0 | 0 | 10 | 10 | 0 | 3 | 0 | 4.0 |
| 13 | 7.5 | 0 | 0 | 0 | 0 | 1 | 0 | 35.7 | 4.0 |
| 14 | 7.5 | 0 | 0 | 0 | 0 | 1 | 3 | 28.1 | 4.0 |
| 15 | 7.5 | 0 | 0 | 1 | 10 | 1 | 0 | 0.0 | 4.0 |
| 16 | 7.5 | 0 | 0 | 1 | 10 | 1 | 3 | 0.0 | 4.0 |

| | |
|---|---|
| 17 | Media |
| 18 | Triton X |

The results show that the TER reduction was observed with all formulations. Media applied to the apical side did not reduce TER whereas Triton X treated group showed significant TER reduction as expected.

### EXAMPLE 3

### Cell Viability and Cytotoxicity

Cell viability was assessed using the MTT assay (MTT-100, MatTek kit). Thawed and diluted MTT concentrate was pipetted (300 μl) into a 24-well plate. Tissue inserts were gently dried, placed into the plate wells, and incubated at 37° C. for 3 hours. After incubation, each insert was removed from the plate, blotted gently, and placed into a 24-well extraction plate. The cell culture inserts were immersed in 2.0 ml of the extractant solution per well (to completely cover the sample). The extraction plate was covered and sealed to reduce evaporation of extractant. After an overnight incubation at room temperature in the dark, the liquid within each insert was decanted back into the well from which it was taken, and the inserts discarded. The extractant solution (200 μl in at least duplicate) was pipetted into a 96-well microtiter plate, along with extract blanks. The optical density of the samples was measured at 550 nm on a plate reader.

The amount of cell death was assayed by measuring the loss of lactate dehydrogenase (LDH) from the cells using a CytoTox 96 Cytoxicity Assay Kit (Promega Corp., Madison, Wis.). LDH analysis of the apical media was evaluated. The appropriate amount of media was added to the apical surface in order to total 250 μL, taking into consideration the initial sample loading volume. The inserts was shaken for 5 minutes. 150 μL of the apical media was removed to eppendorf tubes and centrifuged at 10000 rpm for 3 minutes. 2 μL of the supernatant was removed and added to a 96 well plate. 48 uL of media was used to dilute the supernatant to make a 25× dilution. For LDH analysis of the basolateral media, 50 μL of sample was loaded into a 96-well assay plates. Fresh, cell-free culture medium was used as a blank. Fifty microliters of substrate solution was added to each well and the plates incubated for 30 minutes at room temperature in the dark. Following incubation, 50 μl of stop solution was added to each well and the plates read on an optical density plate reader at 490 nm.

The results of the MTT assays showed no significant reduction of cell viability when cells were treated with all formulations. Media applied to the apical side did not show an effect on cell viability whereas the Triton X treated group showed significant reduction of cell viability, as expected. The results of the LDH assays showed no significant cytotoxicity was observed when cells were treated with all formulations. Media control applied to the apical side did not show cytotoxicity whereas Triton X treated group showed significant cytotoxicity, as expected.

### EXAMPLE 4

### Permeation

The ability of various permeation enhancers to improve delivery of PTH transmucosally was tested. To this end, 7.5 mg/ml PTH was combined with various permeation enhancers at pH ˜4.0 and osmolality 220-280 mOsm/kg.

The results of measurements of the PTH permeation in the presence of permeation enhancers showed that PTH permeation significantly increases in the presence of 45 mg/ml Me-β-CD, 1 mg/ml DDPC, and 1 mg/ml EDTA. Various degrees of PTH permeation enhancement were observed in the presence of permeation enhancing excipients. The preservative (NaBz) had no significant impact on PTH permeation.

A formulation containing “non-GRAS” enhancers is exemplified by the combination of 45 mg/ml Me-β-CD, 1 mg/ml DDPC, and 1 mg/ml EDTA. Such formulation may also contain a suitable solvent such as water, a preservative, such as sodium benzoate, chlorobutanol or benzalkonium chloride, and a tonicifier such as a sugar or polyol such as trehalose or a salt such as sodium chloride. Alternatively, the formulation could contain other enhancers including alternative solubilizers, surface-active agents and chelators.

A formulation containing “GRAS” enhancers is exemplified by the combination of 1 mg/mL Tween-80, 100 mg/mL ethanol and 1 mg/ml EDTA. Such formulation may also contain a suitable co-solvent such as water, a preservative, such as sodium benzoate, chlorobutanol or benzalkonium chloride, and a tonicifier such as a sugar or polyol such as trehalose or a salt such as sodium chloride. Alternatively, the formulation could contain other GRAS enhancers including alternative surface-active agents, co-solvents, and chelators.

Yet another formulation containing GRAS enhancers is exemplified by inclusion of 1 mg/mL Tween-80 (polysorbate 80). Such formulation may also contain a suitable co-solvent such as water, a preservative, such as sodium benzoate, chlorobutanol or benzalkonium chloride, and a tonicifier such as a sugar or polyol such as trehalose or a salt such as sodium chloride. Alternatively, the formulation could contain other GRAS enhancers such as alternative surface-active agents.

### EXAMPLE 5

### Stability

A PTH formulation was supplied as a liquid in a bottle for intranasal administration via an actuator. Formulations containing 1-10 mg/mL PTH at pH 4.0-4.5 were tested for “as-sold” stability. “As-sold” stability studies are defined as those studies involving formulation stored within a closed (i.e., capped) bottle, placed at specific storage or accelerated temperature conditions for specified amounts of time. Formulation excipients were selected from the group consisting of PTH; methyl-β-cyclodextrin (Me-β-CD); ethylenediaminetetraacetic acid (EDTA); didecanoylphosphatidyl choline (DDPC); chlorobutanol (CB); sodium benzoate (NaBZ), polysorbate 80, and sorbitol. The initial pH of the formulations was adjusted to pH 4.0 or 4.5 with sodium hydroxide or hydrochloric acid, as necessary. The formulations that were tested are shown in Table 2.

TABLE 2
Composition of Various Intranasal PTH Formulations

| Formulation # | Composition |
|---|---|
| 1 | 1 mg/mL PTH, 5 mg/mL preservative (CB), 45 mg/mL Me-β-CD, 1 mg/mL DDPC, |
| | 1 mg/mL EDTA, 26 mg/mL sorbitol, pH~4.0 |
| 2 | 1.5 mg/mL PTH, 5 mg/mL preservative (CB), 45 mg/mL Me-β-CD, 1 mg/mL |
| | DDPC, 1 mg/mL EDTA, 26 mg/mL sorbitol, pH~4.0 |
| 3 | 2 mg/mL PTH, 5 mg/mL preservative (CB or NaBz), 45 mg/mL Me-β-CD, 1 mg/mL |
| | DDPC, 1 mg/mL EDTA, 16.7 mg/mL sorbitol, pH~4.0 or 4.5 |
| 4 | 3 mg/mL PTH, 5 mg/mL preservative (CB), 1 mg/mL polysorbate 80, 31 mg/mL |
| | sorbitol, pH~4.0 |
| 5 | 4 mg/mL PTH, 5 mg/mL preservative (CB), 1 mg/mL polysorbate 80, 31 mg/mL |
| | sorbitol, pH~4.0 |
| 6 | 5 mg/mL PTH, 5 mg/mL preservative (CB or NaBz), 1 mg/mL polysorbate 80, 27.2 mg/mL |
| | sorbitol, pH~4 |
| 7 | 10 mg/mL PTH, 5 mg/mL preservative (CB or NaBz), 1 mg/mL polysorbate 80, |
| | 27.2 mg/mL sorbitol, pH~4 |

The reported storage conditions for injectable FORTEO® (ingredients: teriparatide, glacial acetic acid, sodium acetate, mannitol, m-cresol, and water) is 2-8° C. for up to 28 days (four weeks). The storage stability of PTH formulations #1, #3, #4, and #7 was monitored at regular intervals by determining the remaining percentage of PTH relative to initial using HPLC. All four formulations used in the stability studies included CB as preservative and were at a pH of 4.0. The results in Tables 3 and 4 show PTH intranasal formulations #1, #3, #4, and #7 may be safely stored at 5° C. and 25° C. for at least four weeks without a significant decrease in stability. Formulations #1, #3, #4, and #7 remained stable for at least 24 weeks when stored at 5° C. Formulation #7 was the most stable of the tested formulations at 5° C. and 25° C. Storage conditions of PTH intranasal formulations at 5° C. for at least 24 weeks is longer than the current recommended storage conditions for injectable FORTEO.

TABLE 3
Percent Stability of PTH Formulations at 5° C.

| | Formulation # (5° C.) | | | |
|---|---|---|---|---|
| Time (weeks) | 1 | 3 | 4 | 7 |
| Initial | 100 ± 1.6 | 100 ± 2.3 | 100 ± 0.4 | 100 ± 2.2 |
| 2 | 101.5 ± 1.1 | 99.8 ± 1.9 | 97.5 ± 0.7 | 100.5 ± 1.3 |
| 4 | 98.1 ± 0.9 | 96.5 ± 3.0 | 100 ± 0.6 | 99.3 ± 2.0 |
| 8 | 96.5 ± 3.2 | 98.2 ± 1.7 | 95.7 ± 1.0 | 95.1 ± 6.6 |
| 12 | 97.4 ± 4.1 | 98.8 ± 2.5 | 97.7 ± 1.5 | 103.3 ± 2.3 |
| 24 | 95.2 ± 0.9 | 94.8 ± 1.2 | 97.3 ± 0.5 | 100.6 ± 2.5 |

TABLE 4
Percent Stability of PTH Formulations at 25° C.

| | Formulation # (25° C.) | | | |
|---|---|---|---|---|
| Time (weeks) | 1 | 3 | 4 | 7 |
| Initial | 100 ± 1.6 | 100 ± 2.3 | 100 ± 0.4 | 100 ± 2.2 |
| 2 | 98.3 ± 1.1 | 98.2 ± 2.3 | 97.5 ± 0.2 | 99.7 ± 1.3 |
| 4 | 96.4 ± 1.6 | 93.2 ± 2.2 | 96.2 ± 2.3 | 97.7 ± 1.3 |
| 8 | 91.1 ± 5.2 | 89.6 ± 8.3 | 90.0 ± 0.4 | 92.8 ± 2.8 |
| 12 | 85.4 ± 7.8 | 89.8 ± 4.0 | 94.5 ± 1.0 | 97.1 ± 1.5 |
| 24 | 80.9 ± 1.0 | 81.7 ± 1.2 | 83.9 ± 1.1 | 87.7 ± 1.6 |

Further characterization of the stability of PTH formulations without buffer was conducted at 30° C. (Table 5), 40° C. (Table 6), and 50° C. (Table 7). The percent PTH remaining from initial was determined at 1, 2, 3, and 4 week timepoints. The 30° C. data without buffer is compared to the injectable formulation data containing buffer from U.S. Pat. No. 6,770,623 (the '623 formulation). The '623 formulation contained 0.1 mg/mL rhPTH (1-34), 50 mg/mL mannitol, 2.5 mg/mL m-cresol, 0.52 mg/mL acetic acid and 0.12 mg/mL sodium acetate. Formulations #1 and #4 without a buffer at 30° C. had stability similar to the '623 formulation with buffer at 30° C. At 50° C., Formulations #1, #3, #4 and #7 have a greater stability than the '623 formulation. Formulation #7 was the most stable compared to other formulations tested at 40° C. and 50° C.

TABLE 5
Percent Stability With and Without Buffer at 30° C.

| | With buffer | | | | Without buffer | |
|---|---|---|---|---|---|---|
| | 20 mM acetate | 10 mM acetate | | | Formulation | Formulation |
| Time (weeks) | (′623) | (′623) | ′623 1 | ′623 2 | # 1 | # 4 |
| Initial | 100 | 100 | 100 | 100 | 100 | 100 |
| 1 | 96 | 94 | 100 | — | 101 ± 4.5 | 114 ± 1.5 |
| 2 | 94 | 92 | 96 | 100 | 73.7 ± 2.0 | 105.5 ± 4.3 |
| 3 | 90 | 93 | 97 | — | 94.7 ± 1.8 | 106.2 ± 1.5 |
| 4 | — | 81 | 96 | 96 | 93.8 | 101.6 |

TABLE 6
Percent Stability of PTH formulations at 40° C.

| | Formulation # (40° C.) | | | |
|---|---|---|---|---|
| Time (weeks) | 1 | 3 | 4 | 7 |
| Initial | 100 ± 1.6 | 100 ± 2.3 | 100 ± 0.4 | 100 ± 2.2 |
| 1 | 90.2 ± 1.3 | 92.9 ± 1.5 | 93.9 ± 0.8 | 96.5 ± 1.6 |
| 2 | 80.7 ± 2.8 | 86.1 ± 1.1 | 83.9 ± 0.8 | 88.0 ± 1.3 |
| 4 | 66.9 ± 1.8 | 70.9 ± 1.6 | 70.3 ± 2.1 | 71.7 ± 2.2 |

TABLE 7
Percent Stability With and Without Buffer at 50° C.

| | Formulatins With buffer | | Formulation # | | | | | |
|---|---|---|---|---|---|---|---|---|
| | | 10 mM | 0.9% | | | | | |
| Time | 20 mM acetate | acetate | NaCl | Water | | | | |
| (weeks) | (′623) | (′623) | (′623) | (′623) | 1 | 3 | 4 | 7 |
| Initial | 100 | 100 | 100 | 100 | 100 ± 1.6 | 100 ± 2.3 | 100 ± 0.4 | 100 ± 2.2 |
| 1 | 84 | 80 | 81 | 74 | 88.9 ± 2.4 | 89.6 ± 3.0 | 88.6 ± 0.2 | 91.6 ± 1.6 |
| 2 | 67 | 71 | 58 | 55 | 76.6 ± 1.8 | 75.9 ± 2.2 | 73.5 ± 0.5 | 76.7 ± 2.9 |
| 4 | — | — | — | — | 54.3 ± 1.2 | 54.5 ± 4.4 | 52.0 ± 0.9 | 56.7 ± 0.8 |

PTH formulations #1 and #4 were also tested for in-use and spray stability at both 5° C. and 30° C. storage temperatures over a 29-day period. Results include % Peptide Recover and % Total Peptide Impurity. “In-use” studies are those in which an actuator is present and the bottles were primed five times initially, and then actuated once daily by hand after subjecting to the storage temperatures. All bottles were returned to the 5° C. and 30° C. stability chamber after 30 minute exposure to room temperature. All bottles were actuated daily, and the actuated samples were collected and stored at −20° C. until scheduled for HPLC measurements. HPLC measurements are scheduled for in-use (i.e., in the bottle with an actuator present) and spray (i.e., measured from the spray produced by the actuator in the bottle) samples at Day 0, Day 8, Day 15, Day 22 and Day 29. The HPLC measurements for stability are shown in Table 8 (% Peptide Recovery) and Table 9 (% Total Impurity).

TABLE 8
In-use and Spray % Peptide Recovery at 5° C. and 30° C.

| Time | Formulation | Formulation | | Formulation |
|---|---|---|---|---|
| Point (days) | #1 | #4 | Formulation #1 | #4 |
| | In-use 5° C. | | Spray 5° C. | |
| 0 | 100.0 | 100.0 | 100.0 | 100.0 |
| 15 | 94.2 | 97.8 | 93.9 | 97.3 |
| 22 | 93.8 | 100.1 | 103.0 | 107.9 |
| 29 | 99.3 | 105.3 | 32.9 | 106.0 |
| | In-use 30° C. | | Spray 30° C. | |
| 0 | 100.0 | 100.0 | 100.0 | 100.0 |
| 8 | 103.3 | 107.0 | 109.7 | 110.6 |
| 15 | 84.7 | 99.3 | 130.8 | 103.8 |
| 22 | 98.8 | 103.0 | 99.6 | 101.9 |
| 29 | 94.3 | 97.8 | 34.7 | 102.3 |

TABLE 9
In-use and Spray Total Peptide Impurity at 5° C. and 30° C.

| Time Point | Formulation | Formulation | Formulation | Formulation | Formulation | Formulation |
|---|---|---|---|---|---|---|
| (days) | #1 | #4 | #1 | #4 | #1 | #4 |
| | As-sold 5° C. | | In-use 5° C. | | Spray 5° C. | |
| 0 | 0.9 | 0.4 | 0.5 | 0.3 | 0.5 | 0.5 |
| 8 | 0.9 | 0.7 | 0.7 | 0.5 | 1.1 | 0.7 |
| 15 | 0.9 | 0.4 | 0.7 | 0.5 | 0.8 | 0.5 |
| 22 | 0.8 | 0.6 | 1.1 | 1.4 | 1.6 | 1.3 |
| 29 | 1.7 | 0.7 | 2.0 | 1.3 | 3.8 | 1.6 |
| | As-sold 30° C. | | In-use 30° C. | | Spray 30° C. | |
| 0 | 0.9 | 0.3 | 0.5 | 0.3 | 0.5 | 0.5 |
| 8 | 1.7 | 1.5 | 2.0 | 1.5 | 3.0 | 1.5 |
| 15 | 1.8 | 1.5 | 1.8 | 1.5 | 3.5 | 2.0 |
| 22 | 4.6 | 3.2 | 4.5 | 3.2 | 5.0 | 3.7 |
| 29 | 6.2 | 5.0 | 6.5 | 5.0 | 15.4 | 5.1 |

As-sold, in-use and spray stability studies showed that Formulation #4 (containing polysorbate 80) was more stable than Formulation #1 (containing EDTA). Further studies confirmed that EDTA alone or in combination with polysorbate 80 was inferior to PTH formulations without EDTA. Formulations with EDTA alone caused precipitation and gelling. When EDTA was added in combination with other excipients an increased instability was observed. Stability studies showed that polysorbate 80 alone and in combination with other excipients enhanced stability. Addition of ethanol to the PTH formulations did not enhance stability.

### EXAMPLE 6

### pH Stability

The following formulations were tested for pH stability (Table 10).

TABLE 10
pH Stability Formulations

| | Conc. (mg/ml) | | | | | | | | | | |
|---|---|---|---|---|---|---|---|---|---|---|---|
| | | | | Polysorbate | | | Glacial | Sodium | | | |
| Diluent | Me-β-CD | DDPC | EDTA | 80 | CB | Sorbitol | acetic acid | acetate | Mannitol | m-Cresol | pH |
| FORTLO ® | 0 | 0 | 0 | 0 | 0 | 0 | 0.41 | 0.1 | 45.4 | 3 | 4.0 |
| Me-β-CD | 45 | 1 | 1 | 0 | 2.5 | 29 | 0 | 0 | 0 | 0 | 4.0 |
| Tween | 0 | 0 | 0 | 1 | 2.5 | 36 | 0 | 0 | 0 | 0 | 4.0 |

Solutions without PTH were first tested by pH titration. All three diluents had a pH value of 4.0 before the pH titration. The pH shifts resulting from the addition of base to the FORTEO®, Me-β-CD and Tween formulations containing 1-4 mg/mL PTH and stored without buffer maintain a pH of 4.0 to 4.2 after at least 8 weeks of storage at 5° C. and 25° C. (Table 11). These data show that the PTH formulation composition stably maintains pH without a buffer.

TABLE 11
pH Stability for Me-β-CD and Tween Formulations at 5° C. and 25° C.

| | pH | | | | | | |
|---|---|---|---|---|---|---|---|
| Formu- | 5° C. | | | 25° C. | | | |
| lations | Initial | 4 weeks | 8 weeks | Initial | 2 weeks | 4 weeks | 8 weeks |
| 1 mg/mL | 4.0 | 4.1 | 4.0 | 4.0 | 4.0 | 4.1 | 4.1 |
| PTH | | | | | | | |
| Me-β- | | | | | | | |
| CD* | | | | | | | |
| 2 mg/mL | 4.0 | 4.0 | 4.0 | 4.0 | 4.0 | 4.1 | 4.0 |
| PTH | | | | | | | |
| Me-β- | | | | | | | |
| CD* | | | | | | | |
| 2 mg/mL | 4.0 | 4.2 | 4.1 | 4.0 | 4.1 | 4.1 | 4.1 |
| PTH | | | | | | | |
| Tween* | | | | | | | |
| 4 mg/mL | 4.0 | 4.1 | 4.1 | 4.0 | 4.1 | 4.1 | 4.1 |
| PTH | | | | | | | |
| Tween* | | | | | | | |

*CB at 2.5 mg/mL

### EXAMPLE 7

### Pharmacokinetics (PK) in Human Subjects

The absorption and safety of the PTH nasal spray formulations (see Example 5, Table 2) of this disclosure were evaluated at two dose levels. The bioavailability of FORSTEO (Eli Lilly UK) given subcutaneously was compared with that of two PTH nasal spray formulations of this disclosure at two dose levels. PTH Nasal Spray will be supplied to the clinic as a liquid in a bottle for intranasal administration via an actuator. For the PK studies, Formulations #3, #6, and #7 included NaBz as the preservative. Formulation #3 had a pH of 4.5, while all other formulations were at pH 4.0.

The PTH solution is provided in a multi-unit dose container to deliver a metered dose of 0.1 mL of drug product per actuation. Hydrochloric acid is added for pH adjustment to meet target pH of 4.0±0.2 or 4.5±0.2, as appropriate. The stability of the formulations was monitored at regular intervals.

This study was a single-site, open-label, active controlled, 5 period crossover, dose ranging study involving 6 healthy male and 6 healthy female volunteers. The commercially available formulation of teriparatide, FORSTEO was the active control. The five study periods were as follows:

Period 1: All subjects received FORSTEO (injection) 20 μg subcutaneously.

Period 2: All subjects received 500 μg intranasal dose of teriparatide, 100 microliter spray of intranasal formulation as described in Example 5, Formulation #6, Table 2.

Period 3: All subjects received 200 μg intranasal dose of teriparatide, 100 microliter spray of intranasal formulation as described in Example 5, Formulation #3 Table 2.

Period 4: All subjects received a 1000 μg intranasal dose of teriparatide, 100 microliter spray of intranasal formulation as described in Example 5, Formulation #7 Table 2.

Period 5: All subjects received a 400 μg intranasal dose of teriparatide, 2×100 microliter spray of intranasal formulation as described in Example 5, Formulation #3 Table 2.

Blood samples for PK were collected at 0 (i.e., pre-dose), 5, 10, 15, 30, 45, 60, 90 minutes and 2, 3, and 4 hours post-dose and analyzed using a validated method. Because the bioassay is fully cross reactive with endogenous PTH(1-84), all data was corrected for pre-dose values. When this correction resulted in a negative post-dose value, all such negative values were set to ‘missing’. Values reported as <LLOQ were set to half LLOQ in order to evaluate PK and change from baseline. Standard pharmacokinetic parameters, including AUCₗₐₛₜ, AUC_{inf}, Cₘₐₓ, t_{1/2}, tₘₐₓ, and Kₑ were calculated using WinNonlin. Intra-subject variability of the pharmacokinetic profiles was evaluated for the test versus the reference using analysis of variance methods. An analysis of variance (ANOVA) was performed based on a 2-period design and incorporating a main effect term for each of the two products under consideration (Snedecor G W and Cochran W G, One-Way Classifications—Analysis of Variance. In: Statistical Methods”, 6^{th} ed.: Iowa State University Press, Ames, Iowa, (1967) pp. 258-98). (Subject (Sequence) was a random effect in the model with all others fixed.) A separate model was created for each dose of teriparatide nasal spray versus the reference. The 90% confidence intervals were generated for the ratio of test dose/reference with respect to Cₘₐₓ, AUCₗₐₛₜ, and AUC_{inf}. These values were natural log (ln)-transformed prior to analysis. The corresponding 90% confidence intervals for the geometric mean ratio were obtained by taking the antilog of the 90% confidence intervals for the difference between the means on the log scale. These analyses were not performed to demonstrate bioequivalence but were for informational purposes only. As a result, no adjustment to the confidence level for each of the paired comparisons was made to account for multiplicity of analysis. This study is hypothesis-generating only. For tₘₐₓ, the analyses were run using Wilcoxon's signed-rank test (Steinijans V W and Diletti E (1983) Eur. J. Clin. Pharmacol. 24:127-36) to determine if differences existed between a given test group and the reference group.

For each subject, the following PK parameters were calculated, whenever possible, based on the plasma concentrations of teriparatide for each test article, according to the model independent approach:

Cₘₐₓ Maximum observed concentration;

tₘₐₓ Time to maximum concentration; and

AUCₗₐₛₜ Area under the concentration-time curve from time 0 to the time of last measurable concentration, calculated by the linear trapezoidal rule.

The following parameters were calculated when the data permitted accurate estimation of these parameters:

AUC_{inf} Area under the concentration-time curve extrapolated to infinity, calculated using the formula:

AUC_{inf}=AUCₗₐₛₜ+Cₜ/Kₑ where Cₜ is the last measurable concentration and Kₑ is the apparent terminal phase rate constant;

Kₑ Apparent terminal phase rate constant, where Kₑ is the magnitude of the slope of the linear regression of the log concentration versus time profile during the terminal phase; and

t_{1/2} Apparent terminal phase half-life (whenever possible), where t_{1/2}=(ln 2)/Kₑ. All data was corrected for pre-dose values. When this correction resulted in a negative post-dose value, all such negative values were set to ‘missing’. Values reported as <LLOQ were set to half LLOQ in order to evaluate pK and change from baseline. Actual (not nominal) sampling times were used in the calculation of all PK parameters.

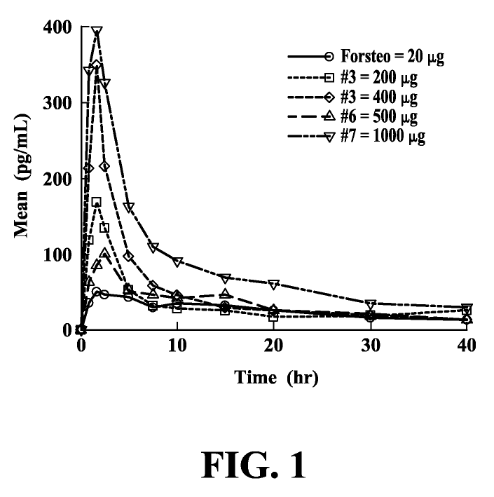

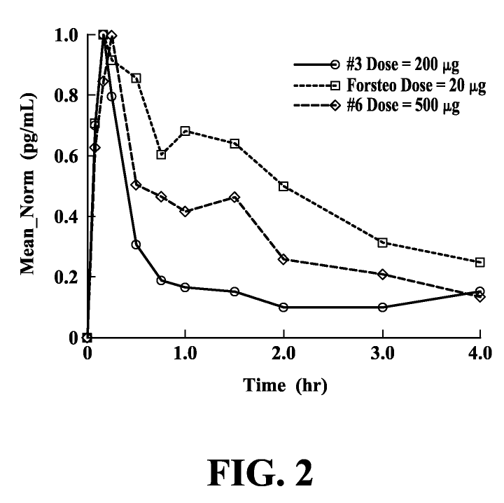

FIGS. 1 and 2 show the mean plasma concentrations versus time for periods 1-5, and the ratio of Cₘₐₓ to mean, low dose formulations versus Forsteo, respectively.

A summary of arithmetic mean pharmacokinetic parameters for each formulation and dose of teriparatide are presented in Table 12. The mean tₘₐₓ was 0.68 versus 0.57 and 0.17 hours for the FORSTEO and low dose nasal formulations of Formulation #6 and #3, respectively. The Cₘₐₓ was 1.6 and 2.4 fold higher than FORSTEO for each low dose formulation. The AUCₗₐₛₜ was 1.23 and 1.45 fold higher than FORSTEO for each low dose formulation.

TABLE 12
Arithmetic Mean Pharmacokinetic Parameters by Formulation and Dose

| | Dose | Tmax | Cmax | AUClast | AUCinf | t½ | Ke |
|---|---|---|---|---|---|---|---|
| Formulation | (μg) | (hr) | (pg/mL) | (hr * pg/mL) | (hr * pg/mL) | (hr) | (1/hr) |
| FORSTEO (injection) | 20 | 0.68 | 70.80 | 85.92 | 132.12 | 1.57 | 0.638 |
| Formulation #6 | 500 | 0.57 | 112.72 | 106.08 | 195.69 | 1.38 | 0.610 |
| Formulation #7 | 1000 | 0.46 | 405.57 | 335.20 | 412.47 | 1.03 | 0.782 |
| Formulation #3 | 200 | 0.17 | 172.72 | 125.07 | 269.60 | 3.10 | 0.720 |
| Formulation #3 | 400 | 0.18 | 349.62 | 206.02 | 238.26 | 1.12 | 1.097 |

In addition, the tₘₐₓ results for each formulation were compared to the FORSTEO control using a simple Wilcoxon signed-rank test. The results (as p-values) are given in Table 13.

TABLE 13
Comparison of Tmax - FORSTEO and Nasal Formulations

| | p-value from Wilcoxon |
|---|---|
| Comparison of Tₘₐₓ | Signed-Rank Test |
| FORSTEO vs. Formulation #6, 500 μg | 0.75 |
| FORSTEO vs. Formulation #7, 1000 μg | 0.53 |
| FORSTEO vs. Formulation #3, 200 μg | 0.10 |
| FORSTEO vs. Formulation #3, 400 μg | 0.24 |

Thus, there does not appear to be differences in the tₘₐₓ values among the formulations with respect to FORSTEO.

The 90% confidence intervals for the comparison of the given formulation and the FORSTEO control for the ratios of Cₘₐₓ, AUCₗₐₛₜ and AUC_{inf} was calculated. The comparisons of each product with FORSTEO were done on a pairwise basis, but no adjustment for multiple testing was included because of the nature of this study.

A summary of clearance rates using the non-compartmental model are presented in Table 14.

TABLE 14
Summary of Clearance Rates

| Formulation | Dose (μg) | Mean (mL/hr) | SD |
|---|---|---|---|
| Formulation #3 | 200 | 1366234.334 | 988398.4 |
| Formulation #3 | 400 | 2527292.583 | 1701658 |
| FORSTEO | 20 | 267446.6298 | 263855.3 |
| Formulation #6 | 500 | 4793716.136 | 4380229 |
| Formulation #7 | 1000 | 3359436.634 | 1665618 |

A summary of percent coefficient of variation for each formulation and dose of teriparatide are presented in Table 15. Based on Cₘₐₓ and AUCₗₐₛₜ, the % CV is lower for Formulation #3 than Formulation #6, Formulation #7 or FORSTEO.

TABLE 15
Percent Coefficient of Variation by Formulation and Dose

| | Dose | Tmax | Cmax | AUClast | AUCinf |
|---|---|---|---|---|---|
| Formulation | (ug) | (hr) | (pg/mL) | (hr * pg/mL) | (hr * pg/mL) |
| FORSTEO | 20 | 165.29 | 51.76 | 66.46 | 62.30 |
| Formulation #6 | 500 | 142.48 | 78.71 | 92.76 | 83.41 |
| Formulation #7 | 1000 | 176.56 | 67.06 | 75.55 | 71.56 |
| Formulation #3 | 200 | 24.72 | 38.78 | 61.55 | 82.28 |
| Formulation #3 | 400 | 21.20 | 48.78 | 55.98 | 68.04 |

A summary of percent relative bioavailability comparing each formulation to the FORSTEO product based on AUCₗₐₛₜ are presented in Table 16. The bioavailability of the Formulation #3 (low and high dose) was 12-15%, whereas Formulations #6 and #7 were approximately 5-8%.

TABLE 16
Relative Bioavailability Compared With FORSTEO
by Formulation and Dose

| | Dose | |
|---|---|---|
| Formulation | (ug) | % Bioavailability |
| Formulation #6 | 500 | 4.9 |
| Formulation #7 | 1000 | 7.8 |
| Formulation #3 | 200 | 14.6 |
| Formulation #3 | 400 | 12.0 |

An exploratory compartmental analysis using WinNonLin 5.0 was conducted to compare the absorption coefficient and elimination coefficient for each formulation. A mixed model analysis of variance on both the Ka and the Ke data, where the subject was included as the random variable was performed, and these results were subanalyzed using the Tukey-Kramer multiple comparison procedure. The individual Ka and Ke data are presented in Table 17. The nasal absorption rates were not significantly different compared to FORSTEO (p=0.50), however the elimination rate for high dose nasal Formulation #3 was significantly faster (p=0.02) than FORSTEO. This is also observed when looking at the ratio of mean Cₘₐₓ to each individual time point per low dose formulation.

TABLE 17
Absorption Coefficient and Elimination Coefficient for Each Formulation

| | | Dose | | Mean | | |
|---|---|---|---|---|---|---|
| Coefficient | Formulation | (μg) | N | (1/hr) | SD | CV % |
| Ka | FORSTEO | 20 | 11 | 11.99 | 7.00 | 58.34 |
| Ka | Formulation #6 | 500 | 8 | 6.95 | 4.83 | 69.46 |
| Ka | Formulation #7 | 1000 | 7 | 10.43 | 7.49 | 71.81 |
| Ka | Formulation #3 | 200 | 6 | 11.02 | 5.29 | 48.05 |
| Ka | Formulation #3 | 400 | 7 | 8.81 | 3.19 | 36.27 |
| Ke | FORSTEO | 20 | 11 | 1.04 | 0.86 | 83.50 |
| Ke | Formulation #6 | 500 | 8 | 1.40 | 1.70 | 121.57 |
| Ke | Formulation #7 | 1000 | 7 | 1.83 | 2.50 | 136.49 |
| Ke | Formulation #3 | 200 | 6 | 2.74 | 2.24 | 81.85 |
| Ke | Formulation #3 | 400 | 7 | 4.08 | 2.35 | 57.69 |

Based on the pharmacokinetic parameters, both nasal formulations had a greater Cₘₐₓ and AUC as compared to FORSTEO. The tₘₐₓ occurred sooner after dosing for the nasal formulations, particularly for Formulation #3. The absorption rates were not significantly different among the nasal and subcutaneous formulations (p=0.5), but elimination rates were faster particularly for the low dose Formulation #3 (p=0.02). However, a t_{1/2} of approximately 1 hour was very similar for the nasal formulations compared to FORSTEO, except for the low dose Formulation #3, where there may be an apparent outlier for subject numbers 1 and 5. If the two subjects are removed the t_{1/2} is 1.5 hours, the same as FORSTEO. The apparent difference in elimination rates may reflect slower wash-in for the subcutaneous product and Formulations #6 and #7 when compared with Formulation #3.

Both nasal formulations have very similar t_{1/2} compared to FORSTEO. Formulation #3 also showed good dose linearity from 200 to 400 μg dose based on the clearance rate and regression analysis. In addition, Formulation #3 was less variable than Formulations #6 and #7 and FORSTEO based on % coefficient of variation. Accordingly, the intranasal formulations of this disclosure exceed the Cₘₐₓ and AUC values for the currently marketed subcutaneous product. This demonstrates that the levels of the marketed product can be exceeded by a nasally administered product, and also that the concentrations of PTH in nasal formulations can be decreased if it is desired to more closely approximate the plasma concentrations of the currently approved product.

Based on the results, nasal administration was less variable than subcutaneous administration and offered a more convenient and compliant route of delivery. Although there were 8 reports of mild post-dose nasal discomfort, there were no findings of irritation, bleeding, etc. at the post-study nasal exam on day 5 of the study. Further, in subsequent studies NaBz preservative was replaced with CB, and post-dose nasal discomfort was not reported with the CB containing formulations. The use of CB as preservative is preferred to avoid nasal discomfort following intranasal administration of PTH formulations.

### EXAMPLE 8

### Droplet Size and Spray Characterization

The droplet size and spray characterization of two teriparatide intranasal formulations (see Example 5, Table 2) were evaluated using the Pfeiffer 0.1 ml Nasal Spray Pump 65550 with 36 mm dip tube. The droplet size distribution is characterized by laser diffraction using a Malvern MasterSizer S modular particle size analyzer and a MightyRunt automated actuation station. Single spray droplet distribution is volume weighted measurement. The Spray Pattern is characterized using a SprayVIEW NSP High Speed Optical Spray Characterization System and SprayVIEW NSx Automated Actuation System. The data are shown in Table 18. The diameter of droplet for which 50% of the total liquid volume of sample consists of droplets of 30 micron and 294 micron for formulation #5 and #2, respectively. There are 3% and 1% of the total liquid volume for formulation #5 and #2, respectively, where the droplet size is less than 10 micron. The ellipticity ratio is 1.3 and 1.4 for formulation #5 and #2, respectively.

TABLE 18
Droplet Size and Ellipticity Ratio for Teriparatide
Intranasal Formulations

| | | | | % <10 | |
|---|---|---|---|---|---|
| | | | | micro- | Ellipticity |
| | D(v, 0.1) | D(v, 0.5) | D(v, 0.9) | meter | Ratio |
| Formulation | 14 | 30 | 65 | 3 | 1.3 |
| #5 | | | | | |
| Formulation | 25 | 294 | 676 | 1 | 1.4 |
| #2 | | | | | |

The spray characteristics and drug purity of PTH formulations were compared as actuated from two nasal pump models made by two manufacturers [Pfeiffer (SAP #65550) vs. Valois (Model Equadel™ 100)]. Two formulations were tested in this study, Formulations #2 and #5 (see Example 5, Table 2). A set of placebos (without drug) was included in all spray experiments as controls. Six vials for each group were provided for spray characterization tests. These vials were prepared and held at 5° C. until ready for the tests. Three of the six vials from each group were concurrently tested and evaluated for Droplet Size Distribution and Pump Delivery parameters.

The results of the comparison are shown in Tables 19 and 20.

TABLE 19
Comparison of Droplet Size for Different Actuators

| | | | | | |
|---|---|---|---|---|---|
| Actuator system | D10 | D50 | D90 | Span | % <10 □m |

Formulation # 5

| | | | | | |
|---|---|---|---|---|---|
| Pfeiffer | 14 | 30 | 65 | 2 | 3.15 |
| Valois | 20 | 52 | 114 | 2 | 0.72 |

Formulation #5 w/o PTH (0 mg/ml PTH)

| | | | | | |
|---|---|---|---|---|---|
| Pfeiffer | 14 | 29 | 62 | 2 | 3.55 |
| Valois | 20 | 50 | 108 | 2 | 0.79 |

Formulation # 2

| | | | | | |
|---|---|---|---|---|---|
| Pfeiffer | 25 | 294* | 676* | 2 | 1.06 |
| Valois | 24 | 67 | 255 | 3 | 0.85 |

Formulation #2 w/o PTH (0 mg/ml PTH)

| | | | | | |
|---|---|---|---|---|---|
| Pfeiffer | 26 | 252* | 610* | 3 | 1.09 |
| Valois | 24 | 67 | 244 | 3 | 0.94 |

*actuation produced bubbles that interfered with the measurement

TABLE 20
Comparison of Ellipticity Ratio for Different Actuators

| Ellipticity Ratio | Pfeffier | Valois |
|---|---|---|
| Formulation #5 | 1.3 | 1.1 |
| Formulation #5 w/o PTH | 1.1 | 1.1 |
| (0 mg/ml PTH) | | |
| Formulation #2 | 1.4 | 1.1 |
| Formulation #2 w/o PTH | 1.4 | 1.1 |
| (0 mg/ml PTH) | | |

### EXAMPLE 9

### Administration of Synthetic and Recombinant PTH₁₋₃₄ Increases Bone Mass in Rats

The anabolic effects of synthetic human PTH₁₋₃₄ and recombinant PTH₁₋₃₄ (Forteo®, Eli Lilly U.S.) were studied in male rats. A common vehicle (composed of glacial acetic acid, m-cresol, sterile water, sodium acetate and mannitol) was used for each treatment Group and Vehicle control.

Experimentally naïve, 5 week old, male Sprague Dawley rats received either vehicle or one of two dose levels (16 μg/kg/d or 80 μg/kg/d) of synthetic or recombinant PTH₁₋₃₄ via subcutaneous (SQ) administration. The animals were randomized into treatment groups (10 rats/group) based on body weight. Each animal was given once daily subcutaneous injections of vehicle or test PTH₁₋₃₄ treatment, starting on Day 1 and continuing for 21 consecutive days. Cage side observations were performed twice daily, and weekly body weight measurements were taken throughout the study. Animals were given a total of two doses of calcein, one dose six (6) and one dose two (2) days prior to scheduled necropsy. On Day 21, blood samples for pharmacokinetic analysis were collected from animals in select treatment groups. At the conclusion of the treatment period and after blood collection on Day 21, the animals were euthanized and bone specimens collected. The treatment groups are shown in Table 21.

TABLE 21
Treatment groups for bone mass study

| | | Dose Level | | |
|---|---|---|---|---|
| Group | Treatment | (μg/kg/d) | Route and Days of Dosing | Group Size |
| 1 | Vehicle | 0 | SQ, 1X/d, Days 1-21 | 10 |
| 2 | Synthetic PTH₁₋₃₄ | 16 | SQ, 1X/d, Days 1-21 | 10 |
| 3 | Recombinant PTH₁₋₃₄ | 16 | SQ, 1X/d, Days 1-21 | 10 |
| 4 | Synthetic PTH₁₋₃₄ | 80 | SQ, 1X/d, Days 1-21 | 10 |
| 5 | Recombinant PTH₁₋₃₄ | 80 | SQ, 1X/d, Days 1-21 | 10 |

Bone in the distal and midshaft regions of the right femur were analyzed using peripheral quantitative computed tomography (pQCT) and bone strength was determined via three-point bending at the femoral mid-shaft and in the marrow cavity of the distal femur. The entire right tibia was subject to dual X-ray absorptiometry scan (DXA).

All animal weights increased over the course of the study. There was no statistically significant difference in body weight between the treatment groups. Bone mineral content, area, and density of four areas of the tibia were analyzed separately (whole tibia and distal, midshaft and proximal tibia) by DXA.

Administration of both forms of human PTH₁₋₃₄ resulted in significant increases in bone mineral content and density at each of the sites examined compared to vehicle control. The increases in bone mineral density were accompanied by increased bone strength at the femoral shaft and trabecular bone in the marrow cavity of the distal femur. The increases in bone mass and strength were dose-dependent. There was no significant difference in bone response between synthetic and recombinant forms of PTH₁₋₃₄ at either of the two doses tested, 16 and 80 μg/kg/d.

These studies confirm that synthetic and recombinant forms of human PTH₁₋₃₄ exhibited comparable anabolic action on bone.

### EXAMPLE 10

### Anabolic Actions and Toxicity Results for Intranasal Administration of PTH₁₋₃₄ in Rats

Toxicity and toxicokinetics of PTH₁₋₃₄ formulations were evaluated in male and female Crl:CD(SD) rats. PTH₁₋₃₄ (synthetic form) was administered once daily via intranasal instillation to rats for at least 13 weeks. For comparison, one group received commercially available recombinant PTH₁₋₃₄ via subcutaneous injection. Assessment of toxicity was based on mortality, clinical observations, ophthalmic examinations, body weights, food consumption, clinical and anatomic pathology, and toxicokinetic evaluations. Two synthetic PTH₁₋₃₄ formulations were used in the study, PTH-072-1 and PTH-074 at low and high doses (formulations are shown in Table 22).

TABLE 22
Intranasal formulations for PTH-072-1 and PTH-074-1

| Formulation | PTH(1-34) | Me-β-CD | DDPC | EDTA | Sorbitol | Polysorbate | CB |
|---|---|---|---|---|---|---|---|
| ID | (mg/ml) | (mg/ml) | (mg/ml) | (mg/ml) | (mg/ml) | 80 (mg/ml) | (mg/ml) |
| Low-PTH- | 2.0 | 45 | 1 | 1 | 26 | 0 | 5 |
| 072-1 | | | | | | | |
| High-PTH- | 4.0 | 45 | 1 | 1 | 26 | 0 | 5 |
| 072-1 | | | | | | | |
| Low-PTH- | 4.0 | 0 | 0 | 0 | 31 | 1 | 5 |
| 074-1 | | | | | | | |
| High-PTH- | 10.0 | 0 | 0 | 0 | 31 | 1 | 5 |
| 074-1 | | | | | | | |

Doses in rats were determined for body weight, body surface area, and nasal surface area. Representative concentrations of PTH₁₋₃₄ for clinical studies were considered to be 1.5 mg/mL and 3.0 mg/mL (and a dose volume of 100 μL). For the lower concentration, a 70 kg human would receive a dose of 2.1 μg/kg based on body weight. At the higher dose a human would receive a dose of 4.3 μg/kg based on body weight. The rat study groups are shown in Table 23.

TABLE 23
Study Groups for Rat Toxicity and Toxiokinetic Studies

| | | | |
|---|---|---|---|
| | No. of | | |
| | Animals | | |
| | Male/ | Dose Level | |
| Group | Female | (μg/kg/day) | Mode of Administration |

Toxicity Animals

| | | | |
|---|---|---|---|
| 1 Control (placebo)^{†} | 10/10 | 0 | Intranasal 50 μL/kg/dose |
| 2 Low - PTH-072-1 | 10/10 | 100 | Intranasal 50 μL/kg/dose |
| 3 High - PTH-072-1 | 10/10 | 200 | Intranasal 50 μL/kg/dose |
| 4 Low - PTH-074-1 | 10/10 | 200 | Intranasal 50 μL/kg/dose |
| 5 High - PTH-074-1 | 10/10 | 500 | Intranasal 50 μL/kg/dose |
| 6 PTH₁₋₃₄ Injection | 0/10 | 25 | Subcutaneous 0.312 mL/kg |

Toxicokinetic Animals

| | | | |
|---|---|---|---|
| 7 High - PTH-072-1 | 10/10^{‡} | 200^{‡} | Intranasal 50 μL/kg/dose |
| 8 High - PTH-074-1 | 10/10^{‡} | 500^{‡} | Intranasal 50 μL/kg/dose |
| PTH₁₋₃₄ Injection | 0/10 | 25^{‡} | Subcutaneous 0.312 mL/kg |

^{†}Placebo was 0.9% Sodium Chloride, USP (sterile saline).
^{‡}Four animals/sex from Groups 7 and 8 and four females in Group 9 received Calcein (10 mg/kg via intraperitoneal injection) on Days 86 and 90.

The t_{1/2} for PTH₁₋₃₄ when administered in the PTH-072-1 formulation ranged from 14 to 21 minutes in male and female rats; Tₘₐₓ ranged from 5 to 15 minutes for both males and females. Cₘₐₓ ranged from 5,041 pg/mL to 12,911 pg/mL in male rats and from 3,044 pg/mL to 5106 pg/mL in female rats. AUCₗₐₛₜ ranged from 100,038 pg·min/mL to 457,644 pg·min/mL in males and 58,890 pg·min/mL to 73,444 pg·min/mL in females. In comparison to a clinical study with PTH-072-1 formulation, the AUCₗₐₛₜ values for male and female rats exceeded that in humans by 80-fold and 13-fold, respectively.

The t_{1/2} for PTH₁₋₃₄ when administered in the PTH-074-1 formulation ranged from 12 to 24 minutes; Tₘₐₓ ranged from 5 to 30 minutes for both male and female rats. Cₘₐₓ ranged from 12,251 pg/mL to 35,964 pg/mL in male rats and from 3,679 pg/mL to 17,175 pg/mL in female rats. AUCₗₐₛₜ ranged from 252,790 pg·min/mL to 1,010,348 pg·min/mL in males and 78,059 pg·min/mL to 377,278 pg·min/mL in females. In comparison to a clinical study with PTH-074-1 formulation, the AUCₗₐₛₜ values for male and female rats exceeded that in humans by 71-fold and 27-fold, respectively.

The t_{1/2} for PTH₁₋₃₄ when administered by injection ranged from 15 to 23 minutes; Tₘₐₓ was 5 minutes for female rats. Cₘₐₓ and AUCₗₐₛₜ ranged from 7,721 pg/mL to 12,200 pg/mL and from 140,945 pg·min/mL to 296,908 pg·min/mL, respectively.

The t_{1/2} and Tₘₐₓ for PTH₁₋₃₄ was similar among the intranasal groups and subcutaneous dose group. Cₘₐₓ and AUCₗₐₛₜ were higher in male rats than female rats, which was an anticipated result for PTH₁₋₃₄. Bioavailability appeared slightly greater in the PTH-072-1 formulation. The highest dose for each formulation exceeded the doses anticipated for clinical evaluation of PTH₁₋₃₄ via intranasal administration in humans. For nasal surface area, the dose multiples were approximately 5-fold or greater in the rat. Based on body surface area or body weight, dose multiples in the rat were approximately 17-fold or 95-fold or greater, respectively. These pharmacokinetics results confirm that the doses selected were sufficient to evaluate the nasal and systemic toxicology of PTH₁₋₃₄ when administered via intranasal instillation.

No PTH₁₋₃₄ related clinical signs, ophthalmic observations, body weight changes, or food consumption changes were observed, regardless of route of administration, dose level, or formulation. No changes considered to be attributable to the intranasal administration of PTH₁₋₃₄ were observed in the nasal turbinate tissues from any animal in the study. The nasal cavity was sectioned such that meaningful regions of the cavity were represented, and the soft (epithelial lining) or hard (bone and cartilage based structures) tissues of the nasal cavity were examined.

Evaluation of trabecular bone in sternum and femur did not reveal any effects that were considered to be adverse. Rather, changes in trabecular bone revealed observations consistent with the anabolic actions of PTH₁₋₃₄. Observations of thickened trabecular bone in the femur and sternum were noted for females dosed with 25 μg/kg/day SQ and 200 μg/kg/day PTH-072-1 or 500 μg/kg/day PTH-074-1 intranasally. Females in the low dose intranasal PTH₁₋₃₄ groups, 100 and 200 μg/kg/day PTH-072-1 and PTH-074-1 were similar to control females. Trabecular bone in femur and sternum was thickened in male animals dosed intranasally with either PTH₁₋₃₄ formulation. The thickening was observed in males given PTH₁₋₃₄ at 500 μg/kg/day and 200 μg/kg/day in the PTH-074-1 formulation; and males given 200 μg/kg/day in the PTH-072-1 formulation. The low dose (100 μg/kg/day) males for PTH-072-1 were similar to controls. The anabolic effect was greater in males compared to females at the corresponding intranasally administered dose.

### Summary

No observations in animal health, clinical pathology, or tissue/organ morphology were found that indicate unexpected toxicologic results for the intranasal instillation of PTH₁₋₃₄. There were no observational differences between the animals that received PTH₁₋₃₄ via intranasal instillation compared to those dosed via subcutaneous injection. Examination of multiple sections representing the entire cavity and representative tissue types indicated once daily intranasal administration of PTH₁₋₃₄ at high doses (and concentrations) was well tolerated. Further, changes in trabecular bone following intranasal PTH₁₋₃₄ administration showed observations consistent with the anabolic actions of PTH₁₋₃₄.

### EXAMPLE 11

### Anabolic Actions and Toxicity Results for Intranasal Administration of PTH₁₋₃₄ in Dogs

Toxicity and toxiokinetics of PTH₁₋₃₄ was studied after administration of PTH₁₋₃₄ once daily by intranasal instillation to dogs for at least 13 weeks. One additional group received recombinant PTH₁₋₃₄ by subcutaneous injection for comparison.

Male and female beagles were assigned among six study groups. Animals assigned to groups 1 through 5 received an intranasal installation of a negative control (0.9% Sodium Chloride for Injection, USP), 40 or 80 μg/kg of body weight/day (μg/kg/day) PTH₁₋₃₄ (synthetic form) in the PTH-072 formulation (Example 10, Table 22), or 80 or 200 μg/kg/day PTH₁₋₃₄ (synthetic form) in the PTH-074-1 formulation (Example 10, Table 22). The dog study groups are shown in Table 24.

TABLE 24
Study Groups for Dog Toxicity and Toxiokinetic Studies

| | No. of | Dose | |
|---|---|---|---|
| | Animals | Level | |
| | Male/ | (μg/ | |
| Group | Female | kg/day) | Mode of Administration |
| 1 Control (placebo) | 4/4 | 0 | Intranasal 0.020 mL/kg/dose |
| 2 Low - PTH-072-1 | 4/4 | 40 | Intranasal 0.020 mL/kg/dose |
| 3 High - PTH-072-1 | 4/4 | 80 | Intranasal 0.020 mL/kg/dose |
| 4 Low - PTH-074-1 | 4/4 | 80 | Intranasal 0.020 mL/kg/dose |
| 5 High - PTH-074-1 | 4/4 | 200 | Intranasal 0.020 mL/kg/dose |
| 6 PTH₁₋₃₄ Injection | 4/4 | 6 | Subcutaneous 0.081 mL/kg |
| | | | (Days 1-40) or 0.075 mL/kg |
| | | | (Days 41-92) |

For PTH-072-1 formulations, Tₘₐₓ for PTH₁₋₃₄ ranged from 8 to 26 minutes. Cₘₐₓ and AUCₗₐₛₜ showed dose-dependence. For PTH-074-1 formulations, Tₘₐₓ for PTH₁₋₃₄ ranged from 8 to 24 minutes. Following subcutaneous injection of PTH₁₋₃₄ Tₘₐₓ for PTH₁₋₃₄ ranged from 13 to 26 minutes. Systemic exposure for subcutaneous injection, as determined by Cₘₐₓ, AUC₁ₐₛₜ, and AUC_{inf}, were intermediate between the low and high doses of PTH₁₋₃₄ following intranasal administration.

The relative bioavailability for PTH₁₋₃₄ was greater at the higher concentration dose for both intranasal formulations. The relative bioavailability for PTH₁₋₃₄ was greater in the PTH-072-1 formulation. The Tₘₐₓ, Cₘₐₓ, and AUCₗₐₛₜ for PTH₁₋₃₄ in each formulation were consistent with achieving peak levels soon after dosing and returning to baseline within a few hours post-dose; this general profile is desired for induction of anabolic actions of PTH₁₋₃₄.

In comparison to clinical doses, for the low dose intranasal formulations nasal surface doses were approximately 0.9-fold for Day 1 and at least 1.5-fold by the end of the study. For the high dose intranasal formulations, nasal surface area doses were at least 1.0-fold on Day 1 and 3.8-fold or greater by the end of the study. Cₘₐₓ and AUCₗₐₛₜ for PTH₁₋₃₄ were at least 7-fold and 10-fold, respectively, greater in the dog than that found in humans at representative doses.

Results were collected for mortality, clinical signs, gross nasal passage observations, ophthalmic findings, electrocardiogram measurements, blood pressure and heart rate differences, body weights, food consumption, clinical and anatomic pathology, and toxicokinetic evaluations. All animals in the study survived to scheduled necropsy. No PTH₁₋₃₄ related clinical signs, ophthalmic findings, electrocardiogram differences, blood pressure and heart rate differences, body weights, or food consumption changes were noted. The nasal cavity was sectioned such that meaningful regions of the cavity were represented, and the soft (e.g., epithelial lining) or hard tissues (e.g., bone and cartilage based structures) of the nasal cavity were examined. There were no histologic changes in nasal tissues that were considered to be attributable to the intranasal administration of PTH₁₋₃₄.

Anabolic effects considered to be associated with administration of PTH₁₋₃₄ were reported in dogs administered PTH₁₋₃₄ either intranasally or subcutaneously. The mean total serum calcium for males and females is shown in Table 25. Intranasal administration of PTH₁₋₃₄ in the PTH-072-1 formulations, PTH-074-1 formulations, and subcutaneous injection resulted in a minimal to moderate (>12 mg/dL) increase in serum calcium, which is an expected physiological effect of PTH₁₋₃₄. Increased serum calcium was noted at 2, 4, and 6 hours post-dose with the peak level at 2 or 4 hour time point. PTH₁₋₃₄ injection, but not the intranasal formulations, produced elevated serum calcium levels at the pre-dose time point. The absolute level for group mean serum calcium and the frequency of statistically significant elevation was similar for the injection group and the two high does intranasal formulations, but slightly higher for the injection group. The magnitude of change for the intranasal formulations was dose-dependent. Serum ionized calcium followed the same general pattern as total calcium.

The time and magnitude of the observed effect precludes the likelihood of catabolic effects. Instead, the biodynamic effect is one of an anabolic drug. Such anabolic effects in animals are predictive of resistance to fracture in humans and used as predictors by the FDA.

Transiently elevated serum calcium is an expected action of PTH₁₋₃₄, and there were no adverse clinical observations noted in association with the transient elevation in serum calcium.

TABLE 25
Mean Total Serum Calcium (*P< or =0.05)

| | | | | | | | | | |
|---|---|---|---|---|---|---|---|---|---|
| | Pre- | Dosing | Dosing | Dosing | Dosing | Pre- | Dosing | Dosing | Dosing |
| | dose | (d2) | (d27) | (d27) | (d27) | dose | (d89) | (d89) | (d89) |
| Group | (d7) | 6 hrs | 2 hrs | 4 hrs | 6 hrs | (d89) | 2 hrs | 4 hrs | 6 hrs |

Males (mg/dL)

| | | | | | | | | | |
|---|---|---|---|---|---|---|---|---|---|
| 1 Control | 11.6 ± 0.29 | 11.7 ± 0.15 | 11.7 ± 0.13 | 11.7 ± 0.10 | 11.5 ± 0.17 | 11.7 ± 0.21 | 11.3 ± 0.18 | 11.6 ± | 11.8 ± |
| (placebo | | | | | | | | 0.24 | 0.49 |
| 2 Low - | 11.7 ± 0.26 | 11.8 ± 0.31 | 12.6 ± 0.71 | 12.6 ± 0.53 | 11.8 ± 0.51 | 11.2 ± 0.13 | 12.1 ± 0.45 | 12.1 ± | 11.3 ± |
| PTH-072-1 | | | | | | | | 0.45 | 0.22 |
| 3 High - | 11.5 ± 0.36 | 11.8 ± 0.28 | 13.2* ± 1.02 | 13.4* ± 1.33 | 12.4 ± 0.49 | 11.6 ± 0.38 | 12.7* ± 0.45 | 12.7* ± | 12.1 ± |
| PTH-072-1 | | | | | | | | 0.54 | 1.35 |
| 4 Low - | 11.6 ± 0.30 | 11.9 ± 0.13 | 13.1* ± 0.48 | 13.0 ± 0.66 | 12.1 ± 0.31 | 11.7 ± 0.48 | 12.3* ± 0.40 | 12.0 ± | 11.5 ± |
| PTH-074-1 | | | | | | | | 0.50 | 0.49 |
| 5 High - | 11.8 ± 0.58 | 12.3 ± 0.85 | 14.0* ± 0.99 | 13.8* ± 0.73 | 12.4 ± 0.27 | 11.8 ± 0.30 | 13.1* ± 0.67 | 13.1* ± | 12.1 ± |
| PTH-074-1 | | | | | | | | 0.97 | 0.60 |
| 6 PTH₁₋₃₄ | 11.3 ± 0.29 | 13.4* ± 0.92 | 13.9* ± 0.66 | 14.6* ± 1.00 | 13.4* ± 0.88 | 12.0 ± 0.51 | 13.5* ± 0.39 | 14.3* ± | 13.5 ± |
| Injection | | | | | | | | 0.70 | 0.61 |

Females (mg/dL)

| | | | | | | | | | |
|---|---|---|---|---|---|---|---|---|---|
| 1 Control | 11.5 ± 0.38 | 11.4 ± 0.24 | 11.3 ± 0.15 | 11.6 ± 0.10 | 11.3 ± 0.15 | 11.1 ± 0.24 | 11.2 ± 0.25 | 11.2 ± | 11.2 ± |
| (placebo | | | | | | | | 0.13 | 0.14 |
| 2 Low - | 11.5 ± 0.13 | 11.5 ± 0.25 | 12.6* ± 0.46 | 12.2 ± 0.25 | 11.6 ± 0.29 | 11.1 ± 0.33 | 11.9 ± 0.29 | 11.6 ± | 11.0 ± |
| PTH-072-1 | | | | | | | | 0.17 | 0.38 |
| 3 High - | 11.7 ± 0.29 | 11.8 ± 0.22 | 13.4* ± 0.13 | 13.2* ± 0.38 | 12.2* ± 0.30 | 11.5 ± 0.26 | 12.3* ± 0.29 | 12.4* ± | 11.6 ± |
| PTH-072-1 | | | | | | | | 0.54 | 0.67 |
| 4 Low - | 11.4 ± 0.26 | 11.3 ± 0.17 | 13.0 ± 0.69 | 12.7* ± 0.54 | 11.9 ± 0.40 | 11.4 ± 0.22 | 12.0 ± 0.37 | 11.7 ± | 11.2 ± |
| PTH-074-1 | | | | | | | | 0.30 | 0.17 |
| 5 High - | 11.1 ± 0.38 | 11.9 ± 0.44 | 13.2* ± 0.39 | 13.5* ± 0.79 | 12.5* ± 0.34 | 11.6 ± 0.29 | 12.6 ± 0.64 | 12.5* ± | 11.9 ± |
| PTH-074-1 | | | | | | | | 0.57 | 0.42 |
| 6 PTH₁₋₃₄ | 11.5 ± 0.06 | 13.1* ± 0.46 | 14.1* ± 0.12 | 14.7* ± 0.37 | 13.3* ± 0.34 | 12.1* ± 0.19 | 13.3* ± 0.25 | 13.8* ± | 12.6* ± |
| Injection | | | | | | | | 0.21 | 0.41 |

The (gross) nasal passage examination showed an increased incidence of erythema in PTH₁₋₃₄ treated animals (both subcutaneous and intranasal administration) compared to placebo control. PTH₁₋₃₄ is known to have actions on vascular tone, and erythema is likely a reflection of the pharmacology of PTH₁₋₃₄.

An attenuation of the normal age-related decrease in serum alkaline phosphatase activity is another effect of PTH₁₋₃₄. Mean serum alkaline phosphatase activity dropped approximately 47% and 48% on Day 93 for placebo control males and females, respectively. None of the PTH₁₋₃₄ treated groups (both subcutaneous and intranasal administration) showed a drop of greater than 30% in alkaline phosphatase activity. Attenuation of serum alkaline phosphatase activity was statistically significant in male dogs in both high dose intranasal groups as well as the males in the injection group.

Evaluation of trabecular bone in sternum and femur did not reveal any effects that were considered to be adverse. Rather, changes in trabecular bone revealed observations consistent with the anabolic actions of PTH₁₋₃₄. PTH₁₋₃₄ related changes of minimally thickened trabecular bone in the femur and sternum were observed in dogs dosed subcutaneously or intranasally at the high dose for PTH-072-1 and PTH-074-1.

### Summary

No observations in animal health, clinical pathology, or tissue/organ morphology indicated toxicologic results for the intranasal instillation of PTH₁₋₃₄ with formulations PTH-072-1 or PTH-074-1.

Elevated serum calcium was observed with intranasal doses of PTH₁₋₃₄. The elevated serum calcium is an anabolic effect of PTH. Higher alkaline phosphatase activity in intranasal and subcutaneous PTH₁₋₃₄ treated animals was suggestive of osteoblast activity. A higher incidence of minimally thickened trabecular bone was noted in femur and sternum of PTH₁₋₃₄ treated animals.

The anabolic actions and toxicity studies in both rats and dogs demonstrate that the intranasal route of administration is an effective means for the administration of PTH₁₋₃₄. These results show the safety and efficacy of intranasal administration of the described PTH₁₋₃₄ formulations. Further, the transient increase in serum calcium, higher alkaline phosphatase activity, and thickening of trabecular bone are predictive of the ability of intranasal PTH to increase bone mass, increase bone strength, and decrease the incidence of bone fracture in humans.

### EXAMPLE 12

### In Vitro Effects of Chlorobutanol on Permeation of Intranasal API Formulations

Chlorobutanol (CB) was added to API formulations (PTH and Calcitonin) to test permeation in the MatTek in vitro system. CB was tested at varying concentrations (0, 1.25, 2.5, 3.75 and 5 mg/mL) in 10 mM citrate buffer, pH 4.0 and adjusted for a final target osmolality of 220 mOsm with sorbitol. Calcitonin and PTH were added at 2 mg/mL in each of the respective control or test formulations (n=6 insert per formulation). 1 mL of each formulation was placed into silanized 3 cc vials. A 0.1 mL volume of a 20× stock of one of the API compounds was added to 0.9 mL of 1.1× concentrated diluent form of each of the CB formulations to make the unique formulations. The study design is shown in Table 26. Results of the permeation study are shown in Table 27.

TABLE 26
Study Design for CB Permeation Study

| | CB | | |
|---|---|---|---|
| # | (mg/mL) | API | Testing |
| 6 | 0 | 2 mg/mL Calcitonin | pH, Osm, TER and [Calcitonin] by |
| | | | ELISA |
| 7 | 1.25 | 2 mg/mL Calcitonin | pH, Osm, TER and [Calcitonin] by |
| | | | ELISA |
| 8 | 2.5 | 2 mg/mL Calcitonin | pH, Osm, TER and [Calcitonin] by |
| | | | ELISA |
| 9 | 3.75 | 2 mg/mL Calcitonin | pH, Osm, TER and [Calcitonin] by |
| | | | ELISA |
| 10 | 5 | 2 mg/mL Calcitonin | pH, Osm, TER and [Calcitonin] by |
| | | | ELISA |
| 11 | 0 | 2 mg/mL PTH1-34 | pH, Osm, TER and [PTH1-34] by |
| | | | ELISA |
| 12 | 1.25 | 2 mg/mL PTH1-34 | pH, Osm, TER and [PTH1-34] by |
| | | | ELISA |
| 13 | 2.5 | 2 mg/mL PTH1-34 | pH, Osm, TER and [PTH1-34] by |
| | | | ELISA |
| 14 | 3.75 | 2 mg/mL PTH1-34 | pH, Osm, TER and [PTH1-34] by |
| | | | ELISA |
| 15 | 5 | 2 mg/mL PTH1-34 | pH, Osm, TER and [PTH1-34] by |
| | | | ELISA |

TABLE 27
Permeation Results With Addition of CB

| | Formulation | | API | % | |
|---|---|---|---|---|---|
| ABI | # | CB mg/mL | ug/mL | Permeation | % SD |
| Calcitonin | 6 | 0 | 3.27E−01 | 0.164 | 0.37 |
| | 7 | 1.25 | 3.42E−01 | 0.171 | 0.69 |
| | 8 | 2.5 | 3.42E−01 | 0.171 | 0.97 |
| | 9 | 3.75 | 3.44E−01 | 0.172 | 0.42 |
| | 10 | 5 | 3.51E−01 | 0.175 | 1.01 |
| PTH | 11 | 0 | 2.36E−04 | 0.12 | 8.84 |
| | 12 | 1.25 | 4.80E−04 | 0.24 | 21.32 |
| | 13 | 2.5 | 1.26E−03 | 0.63 | 14.99 |
| | 14 | 3.75 | 2.69E−03 | 1.34 | 8.31 |
| | 15 | 5 | 3.34E−03 | 1.67 | 6.14 |

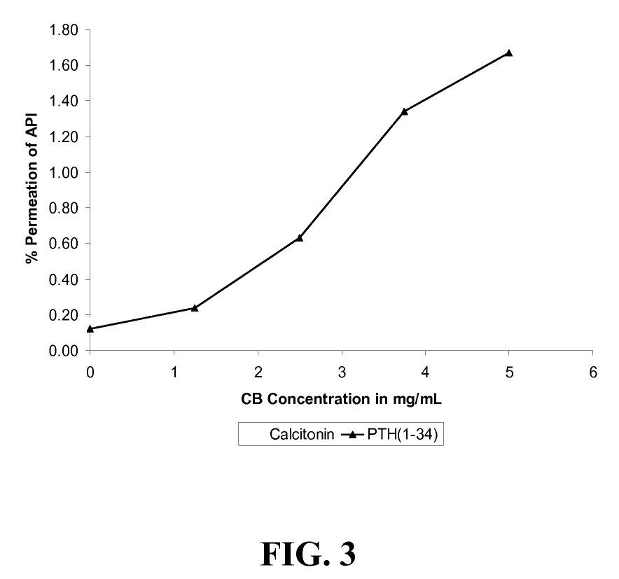

The combined ABI permeation results are shown in FIG. 3. Addition of CB enhanced permeation for PTH. The increase in % permeation of PTH was enhanced with increasing concentration of CB. CB failed to enhance permeation for Calcitonin, therefore, the effect appears to be API-dependent.

### EXAMPLE 13

### In vitro Effects of Chlorobutanol on Intranasal PTH Formulations

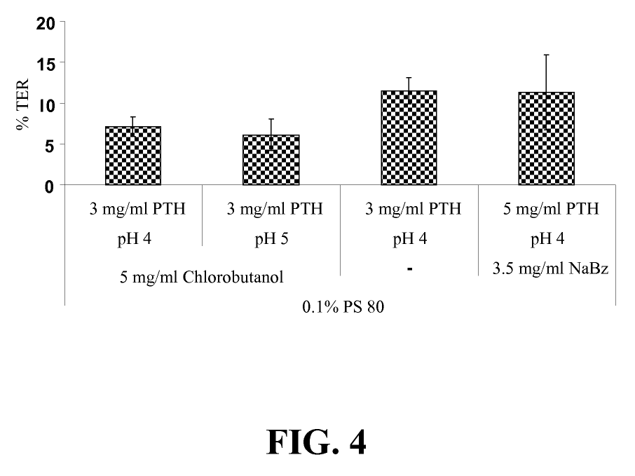

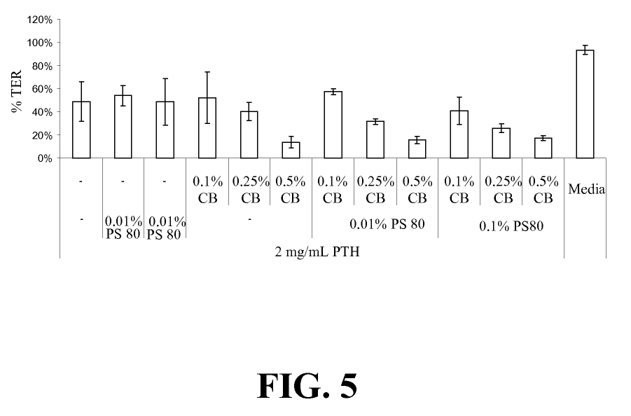

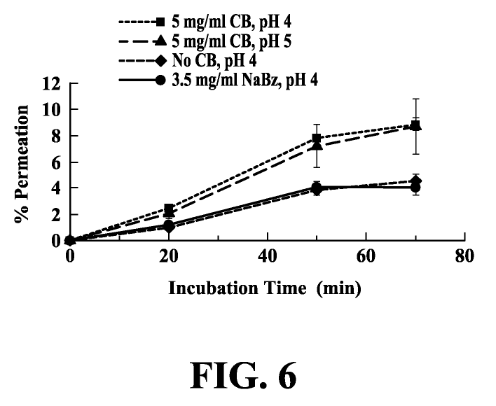

An in vitro study was conducted to determine the effects of chlorobutanol (CB) on intranasal human parathyroid hormone 1-34 (PTH) formulations containing either polysorbate 80 (PS80) or Me-β-CD, DDPC and EDTA (PDF). Addition of CB to the formulations resulted in an increase in TER reduction compared to formulations without preservative or with NaBz (FIG. 4). The reduction in % TER was enhanced in the PS80 formulation (2 mg/mL PTH) with increasing concentration of CB (FIG. 5). Permeation was increased in the PS80 formulations (3 mg/mL PTH) containing CB compared to formulations without preservative or with NaBz (FIG. 6). CB did not appear to effect the % permeation in PDF formulations. The permeation results were similar for the PDF formulations containing CB or NaBz.

The effect of CB on different PS80 formulations was tested. The test formulations are shown in Table 28. A volume of 1 mL was prepared for each formulation. The formulations were prepared from stock solutions of each component (225 mg/mL Me-β-CD, mg/mL DDPC, 5 mg/mL EDTA, 5 mg/mL polysorbate 80, 320 mg/mL sorbitol and 20 mg/mL PTH). The chlorobutanol was added as a solid.

TABLE 28
PTH Formulations Containing Polysorbate 80 and CB

| | Conc. (mg/ml) | | | | | | Cal. |
|---|---|---|---|---|---|---|---|
| Formulation # | PTH | PS80 | CB | Sorbitol | HPMC | pH | Osm. |
| Control PS80 | 3.3 | 1 | 5 | 31 | 0 | 4.0 | 207.0 |
| 0.1 PS80 | 3.3 | 0.1 | 5 | 31 | 0 | 4.0 | 206.3 |
| 0.016 PS80 | 3.3 | 0.016 | 5 | 31 | 0 | 4.0 | 206.3 |
| 0.0016 PS80 | 3.3 | 0.0016 | 5 | 31 | 0 | 4.0 | 206.3 |
| Sorbitol Only | 3.3 | 0 | 5 | 31 | 0 | 4.0 | 206.3 |
| PS80 plus HPMC | 3.3 | 1 | 5 | 31 | 20 | 4.0 | 207.0 |
| PS80 low Osm, | 3.3 | 1 | 5 | 12 | 20 | 4.0 | 100.0 |
| plus HPMC | | | | | | | |

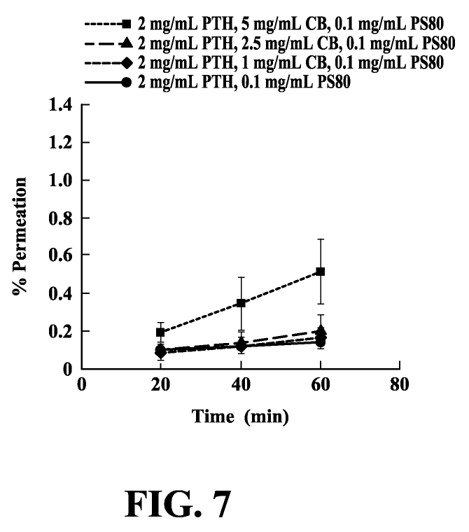

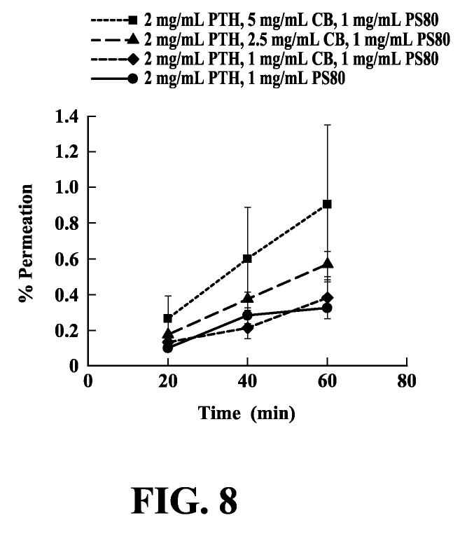

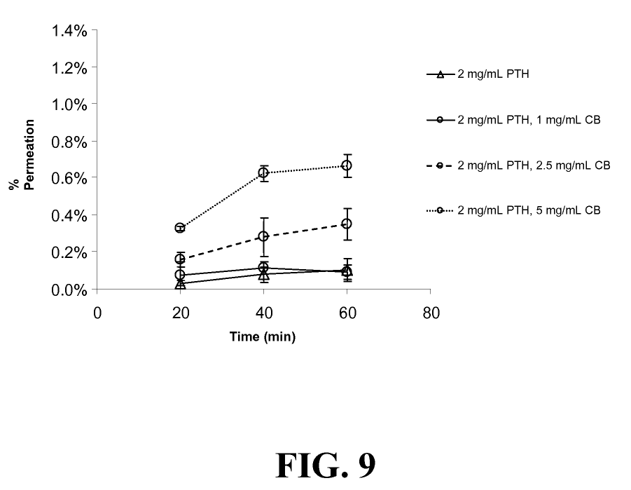

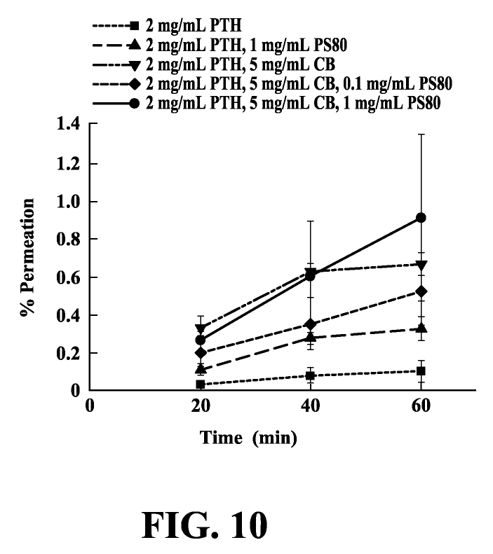

The formulations were checked for pH and osmolality then evaluated in vitro (using a 50 μL insert load volume) for TER and permeation over time (20, 40, 60, 90 min). Each of the formulations was tested in triplicate. The effect of varying concentrations of CB on permeation was tested in the presence of 0.1 mg/mL PS80 (FIG. 7), 1 mg/mL PS80 (FIG. 8), and without PS80 (FIG. 9). Addition of CB to the PTH formulations increased permeation in the presence and absence of PS80. A comparison of the permeation results for PTH containing formulations with and without CB and/or PS80 is shown in FIG. 10. The formulation with the highest permeation contained 2 mg/mL PTH, 5 mg/mL CB, and 1 mg/mL PS80.

PK results from rabbits were also similar among the formulations as shown in Table 29.

TABLE 29
PK Data for PTH Formulations Containing Polysorbate 80 and CB

| | Dose | Tₘₐₓ | Cₘₐₓ | Cₘₐₓ/ | AUCₗₐₛₜ | AUCₗₐₛₜ/ | Relative |
|---|---|---|---|---|---|---|---|
| Formulation # | (ug/kg) | (min) | (pg/mL) | Dose | (pg * min/mL) | Dose | % BA |
| Control PS80 | 50 | 29 | 517 | 10 | 22688 | 454 | NA |
| 0.1 PS80 | 50 | 33 | 475 | 10 | 24452 | 489 | 108 |
| 0.016 PS80 | 50 | 33 | 799 | 16 | 27805 | 556 | 123 |
| 0.0016 PS80 | 50 | 37 | 516 | 10 | 21366 | 427 | 94 |
| Sorbitol Only | 50 | 41 | 868 | 17 | 36825 | 736 | 162 |
| PS80 plus HPMC | 50 | 64 | 404 | 8 | 19466 | 389 | 80 |
| PS80 low | 50 | 23 | 238 | 5 | 11499 | 230 | 51 |
| Osm, plus HPMC | | | | | | | |

### Summary

Intranasal formulations containing PTH and chlorobutanol exhibited dramatically enhanced % permeation. This finding is unexpected because the combination of chlorobutanol with other pharmaceutical peptides, for instance calcitonin, does not enhance drug permeation. Increasing the concentration of CB resulted in increased permeation of PTH. The concentration of chlorobutanol required to increase PTH permeation across the epithelial tissue appears to be at least 0.125% in the aqueous solution containing PTH, more preferably greater than 0.25%, and most preferably greater than 0.5%. The PTH concentration in the aqueous solution can be in the range of 0.02 to 10 mg/mL, more preferably 0.1 to 10 mg/mL, most preferably 1 to 10 mg/mL, in order to achieve the desired drug levels and desired therapeutic effect in a mammal.

### EXAMPLE 14

### In Vitro Preservative Comparison

Five intranasal PTH formulations containing various preservatives (chlorobutanol (CB), sodium benzoate (NaBz), methyl paraben, propyl paraben, or benzalkonium chloride (BAK) were evaluated in the in vitro MatTek cell model system for their effects on transepithelial resistance (TER), cell viability (MTT), cytotoxicity (LDH), and permeation. The compositions of the formulations tested are shown in Table 30.

TABLE 30
Compositions of Formulations With Different Preservatives

| Sam- | Conc. (mg/ml) | | | | | | | | |
|---|---|---|---|---|---|---|---|---|---|
| ple | | | | Methyl | Propyl | | | | Cal. |
| # | PTH | CB | NaBz | paraben | paraben | BAK | Sorbitol | pH | Osm. |
| 1 | 2 | | | | | | 36 | 4.0 | 202 |
| 2 | 2 | 5 | | | | | 31 | 4.0 | 203 |
| 3 | 2 | | 5 | | | | 24 | 4.0 | 206 |
| 4 | 2 | | | 0.33 | | | 36 | 4.0 | 206 |
| 5 | 2 | | | | 0.17 | | 36 | 4.0 | 204 |
| 6 | 2 | | | | | 0.20 | 36 | 4.0 | 204 |

| | | | |
|---|---|---|---|
| 7 | Medium | 4.0 | — |
| 8 | Triton X | 4.0 | — |

### A list of the materials used in the study is shown in Tables 31.

TABLE 31
List of Materials

| Reagent | Grade | Manufacturer | Lot # |
|---|---|---|---|
| PTH | R&D | Bachem | 2500197 |
| Sorbitol | NF | Spectrum | SN0553 |
| Chlorobutanol | NF | Spectrum | UA0237 |
| Methyl Paraben Sodium Salt | NF | Spectrum | VO0560 |
| Propyl Paraben Sodium Salt | NF | Spectrum | UP0798 |
| Sodium Benzoate | NF | Spectrum | TB0355 |
| Benzalkonium Chloride | NF | Spectrum | QE1426 |
| Diluted HCl, 10% w/v | NF | Spectrum | SL0410 |
| Sterile water for irrigation | USP | Spectrum | J5H171 |

The formulations were prepared from stock solutions for the following components, sorbitol, methylparaben, propylparaben, and PTH. The order of addition was sorbitol first, followed by preservatives, and PTH was added in the last step. After PTH was dissolved, the formulations were titrated to pH 4.0 with diluted HCl.

Each formulation was analyzed for pH (Orion 520A+, Nastech ID 0801) and osmolality (Advanced Instruments Inc. Model 2020, loaner osmometer serial #05010095A). The formulations were also evaluated by the in vitro cell assays to determine TER, cell viability, cytotoxicity, and permeation.

Each tissue insert was placed in an individual well containing 1 ml of MatTek basal media. On the apical surface of the inserts, 50 μl of test formulation was applied according to study design, and the samples were placed on a shaker (˜100 rpm) for 1 h at 37° C. The underlying culture media samples were stored at 4° C. for up to 48 hours for LDH (cytotoxicity) and sample permeation (PTH₁₋₃₄ HPLC and enzyme immunoassay (EIA)) evaluations. TER was measured before and after the 1 h incubation. Following the incubation, the cell inserts were analyzed for cell viability via the MTT assay.

The concentrations for permeation time points were determined using enzyme immunoassay (EIA) kits. The EIA kit (p/n S-1178(EIAH6101) was purchased from Peninsula Laboratories Inc. (Division of BACHEM, San Carlos, Calif., 800-922-1516). 17×120 mm polypropylene conical tubes (p/n 352097, Falcon, Franklin Lakes, N.J.) were used for all sample preparations. Eight standards were used for PTH quantitation. The rest of the assay procedure was the same as the kit inserts.

### TER Reduction Effect

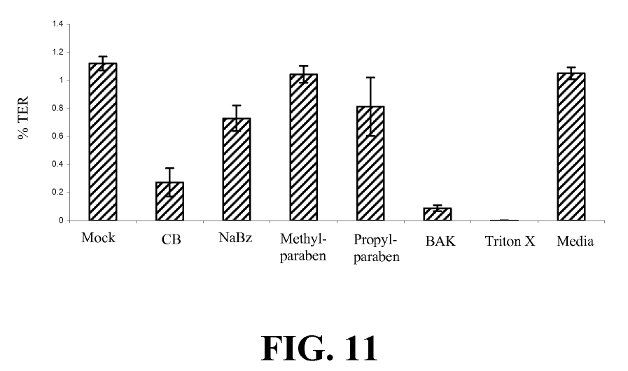

In FIG. 11, “mock” represents the formulation containing only PTH and sorbitol, and serves as the negative control. Both CB and BAK were effective in opening tight junction between the epithelial cells, and resulted in high TER reduction. Slightly lower TER reduction was observed for cells treated with NaBz and propylparaben. Cells treated with methylparaben resulted in similar TER compared with either “mock” or media control, and had no impact on TER of the cells.

### Permeation Enhancement Effect

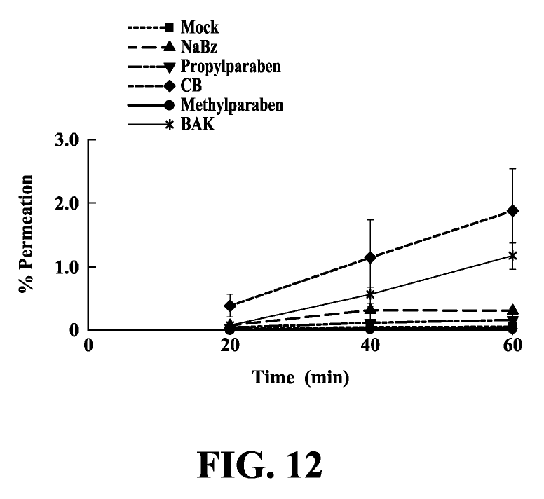

The data in FIG. 12 shows the permeation of various preservative containing formulations at different time points up to 60 minutes after the addition of the formulation to cells. CB and BAK resulted in good % permeation of PTH (1.88% and 1.17% at 60 minutes, respectively).

### MTT Assay (Cell Viability)

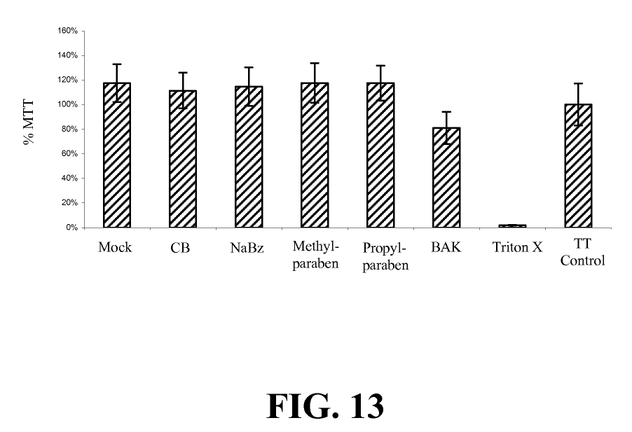

The results in FIG. 13 show the viability of the cells treated with various preservative containing formulations by MTT assay. Cells treated with all formulations except BAK show good cell viability, suggesting no cytotoxicity for those formulations at the preservative concentrations that were tested. BAK at 0.2 mg/mL resulted in a slight cytotoxicity effect on the epithelial cells, and had ˜80.95% of MTT compared with the control.

### LDH Assay

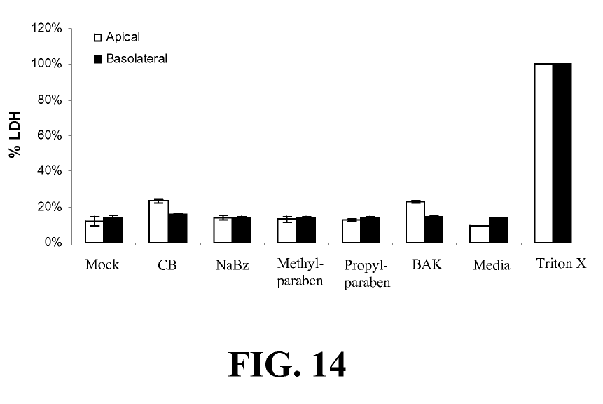

The data in FIG. 14 shows the viability of the cells after treatment with various formulations by LDH assay. Samples from both the apical and basolateral media were assayed for the presence of lactate dehydrogenase. All formulations tested showed a relatively low amount of LDH in the media, suggesting low cytotoxicity to the epithelial cells. Slightly higher LDH was observed for the apical sample from CB and BAK treatments.

### EXAMPLE 15

### In Vivo Effect of Increasing Concentrations of Chlorobutanol on PTH Bioavailability

Pharmacokinetic evaluation of selected intranasal formulations of Teriparatide (Parathyroid Hormone 1-34 [PTH1-34]) following intranasal and subcutaneous dose administration in rabbits was performed to determine the pharmacokinetic parameters for teriparatide in selected formulations containing increasing concentrations of CB. The formulations evaluated in the study are shown in Table 32.

TABLE 32
In vivo Effect of CB Study Design

| | | PTH | CB | PS80 | Sorbitol | |
|---|---|---|---|---|---|---|
| # | Route | (mg/mL) | (mg/mL) | (mg/mL) | (mg/mL) | pH |
| 1 | Intranasal | 3.3 | 0 | 1 | 31 | 4.0 |
| 2 | Intranasal | 3.3 | 2.5 | 1 | 31 | 4.0 |
| 3 | Intranasal | 3.3 | 4 | 1 | 31 | 4.0 |
| 4 | Intranasal | 3.3 | 5 | 1 | 31 | 4.0 |
| 5 | Intranasal | 3.3 | 6 | 1 | 31 | 4.0 |

| | | | |
|---|---|---|---|
| 6 | Subcutaneous | 0.08 | Forteo formulation |

Cₘₐₓ and AUCₗₐₛₜ were determined from the group mean results. Table 33 shows the results of the study.

TABLE 33
Cmax and AUClast for Each Group, and Relative % BA Results.

| # | Cmax | AUClast | % BA* |
|---|---|---|---|
| 1 | 669 | 37515 | 1.6% |
| 2 | 483 | 21405 | 0.9% |
| 3 | 294 | 18375 | 0.8% |
| 4 | 623 | 33295 | 1.4% |
| 5 | 701 | 50678 | 2.2% |
| 6 | 13028 | 753535 | NA |

*Relative to Group 6

The PK rabbit data showed that increasing the concentration of CB in a PS80 formulation resulted in increased % BA of PTH at 6 mg/mL of CB compared to the formulations with lower CB concentrations.

### EXAMPLE 16

### Increased PTH Bioavailability in Humans with Chlorobutanol Containing Formulations

A summary of the results of two human PK studies are shown in Table 34.

TABLE 34
Human PK Results for CB and NaBz Containing Formulations

| | | Dose | AUCₗₐₛₜ | AUCₗₐₛₜ/ | % BA based |
|---|---|---|---|---|---|
| Formulation | Preservative | (ug) | (hr * pg/mL) | Dose | on AUCₗₐₛₜ |
| PS80 | NaBz | 500 | 106.08 | 0.21 | 4.9 |
| PTH 061 | | | | | |
| (#6, Table 2) | | | | | |
| PS80 | NaBz | 1000 | 335.20 | 0.34 | 7.8 |
| PTH 061 | | | | | |
| (#7, Table 2) | | | | | |
| PS80 | CB | 300 | 137.76 | 0.46 | 6.0 |
| PTH 074 | | | | | |
| PS80 | CB | 400 | 298.95 | 0.75 | 9.7 |
| PTH 074 | | | | | |
| PDF | NaBz | 200 | 125.07 | 0.62 | 14.6 |
| 05014 | | | | | |
| (#3, Table 2) | | | | | |
| PDF | NaBz | 400 | 206.02 | 0.51 | 12.0 |
| 05014 | | | | | |
| (#3, Table 2) | | | | | |
| PDF | CB | 100 | 83.13 | 0.83 | 10.9 |
| PTH 072 | | | | | |
| PDF | CB | 150 | 93.77 | 0.62 | 8.2 |
| PK Study 2 | | | | | |
| PTH 072 | | | | | |

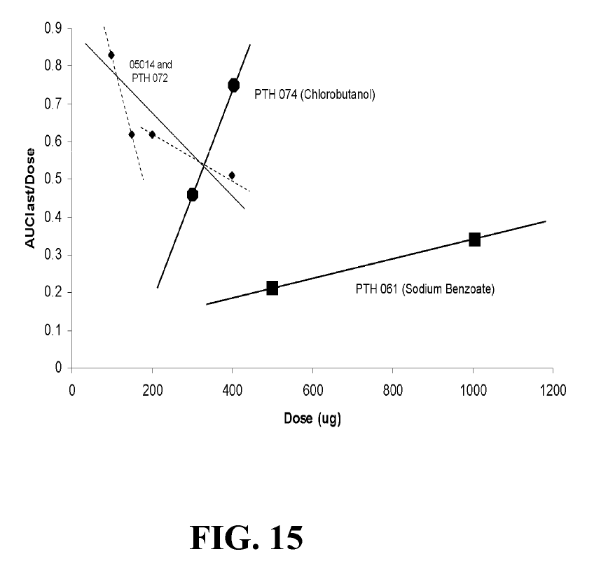

A comparison of the results from the PK studies shows that the presence of CB in the PS80 formulations increases PK compared to formulations with NaBz. FIG. 15 shows a plot of PTH Dose v. AUCₗₐₛₜ/Dose, which illustrates improved PK in PS80 (GRAS) formulations containing CB. The effect is specific to the PS80 formulations, PK in PDF formulations was not improved by using CB instead of NaBz. The above described examples support the use of CB as an enhancer either alone or in combination with PS80.

Although the foregoing disclosure has been described in detail by way of example for purposes of clarity of understanding, it is apparent to the artisan that certain changes and modifications are comprehended by the disclosure and may be practiced without undue experimentation within the scope of the appended claims, which are presented by way of illustration, not limitation.

What is claimed is:

1. An aqueous pharmaceutical formulation for intranasal delivery of PTH, comprising PTH(1-34) and sorbitol.

2. The pharmaceutical formulation of claim 1, further comprising polysorbate 80.

3. The pharmaceutical formulation of claim 1, further comprising chlorobutanol.

4. The pharmaceutical formulation of claim 1, wherein the concentration of PTH(1-34) is at least about 1 mg/ml.

5. The pharmaceutical formulation of claim 1, wherein the concentration of PTH(1-34) is at least about 2 mg/ml.

6. The pharmaceutical formulation of claim 1, wherein the concentration of PTH(1-34) is at least about 6 mg/ml.

7. The pharmaceutical formulation of claim 1, wherein the concentration of PTH(1-34) is at least about 12 mg/ml.

8. The pharmaceutical formulation of claim 3, wherein chlorobutanol is present at less than about 20 mg/mL in the formulation.

9. The pharmaceutical formulation of claim 3, wherein chlorobutanol is present at less than about 10 mg/mL in the formulation.

10. The pharmaceutical formulation of claim 3, wherein chlorobutanol is present at less than about 1 mg/mL in the formulation.

11. The pharmaceutical formulation of claim 1, further comprising hydrochloric acid or sodium hydroxide in an amount sufficient to bring the pH of the formulation in a range from about 3.0 to about 5.0.

12. The pharmaceutical formulation of claim 2, wherein polysorbate 80 is present at less than about 50 mg/mL in the formulation.

13. The pharmaceutical formulation of claim 2, wherein polysorbate 80 is present at less than about 10 mg/mL in the formulation.

14. The pharmaceutical formulation of claim 2, wherein polysorbate 80 is present at less than about 1 mg/mL in the formulation.

15. An aqueous pharmaceutical formulation for intranasal delivery of PTH, comprising PTH(1-34), sorbitol, and a halogenated alkyl alcohol.

16. An aqueous pharmaceutical formulation for intranasal delivery of PTH, comprising PTH(1-34), a polyol, and a chlorobutanol.

17. The pharmaceutical formulation of claim 16, wherein the polyol is selected from the group consisting of sucrose, mannitol, sorbitol, lactose, L-arabinose, D-erythrose, D-ribose, D-xylose, D-mannose, trehalose, D-galactose, lactulose, cellobiose, gentibiose, glycerin, and polyethylene glycol.

18. An aqueous pharmaceutical formulation for intranasal delivery of PTH, comprising PTH(1-34), sorbitol, and surface active agent.

19. The pharmaceutical formulation of claim 18, wherein the surface active agent is selected from the group consisting of nonionic polyoxyethylene ether, polysorbate 80, polysorbate 20, polyethylene glycol, cetyl alcohol, polyvinylpyrolidone, polyvinyl alcohol, poloxamer F68, poloxamer F127, and lanolin alcohol.
